(19) United States
(12) Patent Application Publication
(10) Pub. No.: US 2019/0325996 A1
(43) Pub. Date: Oct. 24, 2019

(54) CLIENT ENGAGEMENT AND CUSTOMER RELATIONSHIP MANAGEMENT (CRM) PLATFORM

(71) Applicant: Kaakpema Yelpaala, Denver, CO (US)

(72) Inventor: Kaakpema Yelpaala, Denver, CO (US)

(21) Appl. No.: 16/455,176

(22) Filed: Jun. 27, 2019

Related U.S. Application Data
(63) Continuation of application No. 15/423,351, filed on Feb. 2, 2017, now abandoned.
(60) Provisional application No. 62/290,432, filed on Feb. 2, 2016.

(51) Int. Cl. G16H 10/60

(52) U.S. Cl. CPC G16H 10/60

(57) ABSTRACT

A client engagement and customer relationship management platform is provided. The client engagement and customer relationship management platform includes a core database including a plurality of patient records, a client engagement and customer relationship management platform application programming interface (API) that is configured to provide access to the core database, one or more interfaces configured to receive a selection of a filter, and provide contact information for a group of patient records located at the core database and accessed via the client engagement and customer relationship management platform API in accordance with the selected filter; and a messaging API configured to receive the contact information for group of patient records, receive a group message, and send the group message to a plurality of patients over a communication network utilizing the contact information.

### CROSS REFERENCE TO RELATED APPLICATION

The present application claims the benefits of and priority, under 35 U.S.C. § 119(e), to U.S. Provisional Application Ser. No. 62/290,432, filed Feb. 6, 2016, entitled “CLIENT ENGAGEMENT AND CUSTOMER RELATIONSHIP MANAGEMENT (CRM) PLATFORM,” which is incorporated herein by reference in its entirety for all that it teaches and for all purposes.

### FIELD OF THE INVENTION

The present disclosure provides a digital health solution that facilitates and enhances practice management and patient engagement.

### BACKGROUND

The current medical diagnostic and treatment environment is populated with traditional medical clinics and establishments consisting of physicians and other medical staff located in central office settings. The customer visits the provider at the central office location for examinations, follow-up appointments, diagnostic tests, and treatment. If other provider opinions or treatments are required, the customer must schedule those appointments separately and subsequently visit those providers at a later date at other locations. Moreover, as medical specialties continue to evolve, the diagnosis and treatment methodology will involve many more providers and specialists than currently accessed. For those customers who have ongoing or complex medical conditions requiring coordinated care, the scheduling and care coordination process for one or more providers is burdensome. This can be a hardship, particularly for those customers located in rural areas or in countries where large distances and/or other obstacles hinder customer's access to medical care.

Accordingly, there is a need to provide a healthcare network that allows patients to receive healthcare anywhere that is accessible by a communications network so that the patient may receive high quality care regardless of their geographical location. Moreover, there is a need to provide a digital health solution that facilitates and enhances practice management and patient engagement.

### SUMMARY

In one embodiment, a client engagement and customer relationship management (CRM) platform is provided. The platform may include one or more systems, including a processor and memory, and may be interconnected to one or more external systems, such as a communication device. For example, the client engagement and customer relationship management platform may provide a digital health solution that facilitates practice management and patient engagement. This practice management solution helps administrators manage patient queues, track payments, and communicate internally. The client engagement and customer relationship management platform also manages patient data through an easy-to-use interface, creating a basic patient record, visit history, and linked family information. Further, the client engagement and customer relationship management platform functions as a patient engagement platform, enabling automated patient appointment and medication reminders, tailored patient communications and bulk health education and campaigns all through SMS text, email, and other forms of communication. For example, U.S. Patent Application Publication No. 2004/0158486 to Nudd et al., which is incorporated herein by reference in its entirety for all that it teaches and for all purposes, discloses a broadcast messaging system to fully leverage the advantages of a healthcare Internet solution that allows for communication between patients and medical providers. Accordingly, Nudd discloses application software that identifies patients who fit certain criteria and emails the selected messages to such patients. In accordance with embodiments of the present disclosure however, in addition to email, SMS, voice, video, or photos may be exchanged between providers and patients. This can be done using WhatsApp, and/or a variety of other services, either 3rd party or custom built services.

In some embodiments, recommendations may be sent to one or more patients and/or patient groups. For example, U.S. Patent Application Publication No. 2014/0278449 to Kharraz Tavakol, which is incorporated herein by reference in its entirety for all that it teaches and for all purposes, discloses applying rules (process logic) and rules data to drive healthcare recommendations to consumers (also referred to as patients). The method allows providers of such rules and recommendations to monitor the consumer responses and further enables each consumer to manage and track his own healthcare actions. In addition (or alternatively) a system and method are provided for delivering recommended healthcare actions that enable the recipient to immediately take action in accordance with the recommendation. These rules provided by Tavakaol and messages based on similar rules may be incorporated into various embodiments described herein in order to provide patient-relevant information to one or more patients. As another example, U.S Patent Application Publication No. 2013/0304496 to Rangadass, which is incorporated herein by reference in its entirety for all that it teaches and for all purposes, discloses integrating electronic records management, patient flow management, hospital operations management and clinical pathway management into an integrated system for managing operations of a medical care facility. In addition to electronic records management, for example, the client engagement and customer relationship management platform keeps the patient in the loop such that the patient may have access to such information in the electronic records, and in some embodiments, messages may be sent to patient groups based on the information in such records.

In some embodiments, the client engagement and customer relationship management platform may include a Patient Queue and Payment Tracker, which captures key check-in/check-out information when a patient arrives/leaves a clinic, hospital, or other location. For example, U.S. Patent Application Publication No. 2007/0226010 to Larsen et al., which is incorporated herein by reference in its entirety for all that it teaches and for all purposes, discloses a system for allowing patients or proxies for those patients to electronically schedule medical resources using a patient interface terminal or interface device such as a kiosk. When a patient accesses a kiosk or interface device to check in for an appointment, in addition to allowing the patient to check in for currently scheduled appointments, the check-in kiosk identifies one or more additional activities that the patient may schedule temporally proximate to the currently scheduled appointments and provides the option to schedule those activities. Here, the additional activities may include existing or unfulfilled orders, routine best practices activities (e.g., a yearly physical for all patients 50 years or older, etc.), prerequisites for one or more of the appointments for which the patient is attempting to check in, etc. This check-in process and similar tools may be incorporated into various embodiments described herein in order to allow a patient to check in and/or check out and capture data regarding the same.

In some embodiments, the client engagement and customer relationship management platform may automate cancellation notifications if appointments are cancelled and automate the capture of data and analytics of key patient flow metrics. Moreover, in some embodiments, the client engagement and customer relationship management platform stores and makes accessible patient records; that is, the client engagement and customer relationship management platform may capture basic health information for a patient at the level of a patient profile and visit history. Further, the client engagement and customer relationship management platform may automate medication reminders and other important health information. In some embodiments, the client engagement and customer relationship management platform may include an appointment manager that includes a calendar per practitioner with patients scheduled in a time slot, thereby automating appointment confirmation messages and appointment reminders, cancellation notifications for any cancelled appointments, and allows users to determine automation criteria for confirmation and scheduling. In some embodiments, the client engagement and customer relationship management platform may provide a group messaging functionally. That is, the client engagement and customer relationship management platform may provide the functionality to create a group based on key filtering criteria and then send a message to the group recipients thereby automating the ability to create a group and automate the sending of messages to the group. In some embodiments, the client engagement and customer relationship management platform is flexible enough to add any additional filter. Some of the filters include, but are not limited to, geolocation, frequency of patient visits, length of time since previous patient visit, family members—is there a mother, father, children, siblings, etc.?—types of payment patient uses, and an amount the patient has spent at the facility. Moreover, the client engagement and customer relationship management platform may automate the updating of groups based on new patient data entered into the system and filtering criteria, automate messages to one or more groups, and automate the insertion of unique codes into a message for a specific group. In some embodiments, the customer engagement and customer relationship management platform may manage practitioner records that include a basic profile of all health workers at the facility and the ability to send messages to those practitioners. In some embodiments, the client engagement and customer relationship management platform may automate messages to practitioners based on a messaging schedule, for example.

In some embodiments, an application may be provided that allows a consumer to locate a healthcare provider; after locating a healthcare provider, they may, from the application, schedule an appointment with the facilities and doctors who are using the client engagement and customer management platform. For example, U.S. Patent Application Publication No. 2011/0009707 to Kaundinya et al., which is incorporated herein by reference in its entirety for all that it teaches and for all purposes, discloses a scheduling tool that allows a client to schedule appointments with one or more providers. This tool and similar tools may be incorporated into various embodiments described herein in order to allow a consumer to schedule an appointment with a healthcare provider.

Similarly, U.S. Patent Application Publication No. 2010/0268549 to Hicks et al., which is incorporated herein by reference in its entirety for all that it teaches and for all purposes, discloses an Internet-based system that provides capabilities for connecting patients with healthcare providers. This system, and similar tools, may be incorporated into various embodiments described herein in order to allow a consumer to find and/or schedule an appointment with a healthcare provider.

In addition, U.S. Patent Application Publication No. 2010/0070303 to Massoumi et al., which is incorporated herein by reference in its entirety for all that it teaches and for all purposes, discloses a booking platform enabling various unaffiliated healthcare practitioner groups to provide, via a centralized aggregator, available healthcare appointments to prospective patients on the aggregator's website. This platform, and similar capabilities may be incorporated into various embodiments described herein in order to allow a consumer to find and/or schedule an appointment with a healthcare provider.

In some embodiments, one or more interfaces may provide healthcare related information to one or more patients. For example, U.S. Patent Application Publication No. 2015/0112696 to Kharraz Tavakol, which is incorporated herein by reference in its entirety for all that it teaches and for all purposes, discloses a system with an interface (e.g., website or mobile application) provided for accessing healthcare appointment availability across multiple disparate sources (e.g., platforms) for the purpose of scheduling patient visits (appointments) and related tasks. These features provided by Tavakaol and similar features may be incorporated into various embodiments described herein in order to allow a consumer to find and/or schedule an appointment with a health care provider for example.

In accordance with embodiments of the present disclosure, a client engagement and customer relationship management platform is provided. The client engagement and customer relationship management platform may include a core database including a plurality of patient records, a client engagement and customer relationship management platform interface configured to provide access to the core database, one or more interfaces configured to receive a selection of a filter and provide contact information for a group of patient records located at the core database and accessed via the client engagement and customer relationship management platform interface in accordance with the selected filter, wherein the group of patient records is defined by the selected filter, and a messaging interface configured to receive the contact information for the group of patient records, receive a group message, and send the group message to one or more communication devices associated with each patient of the group of patients over a communication network utilizing the contact information.

At least one aspect of the client engagement and customer relationship management platform may include an interface application configured to access the core database via the client engagement and customer relationship management platform interface, retrieve patient information from the core database, and store the retrieved patient information at a local storage device of which the interface application resides. At least one aspect of the client engagement and customer relationship management platform may include where the interface application is configured to determine the interface application is offline and store the retrieved patient information in an offline data store. At least one aspect of the client engagement and customer relationship management platform may include where the retrieved patient information is stored in an encrypted form on the local storage device of which the interface application resides. At least one aspect of the client engagement and customer relationship management platform may include where the interface application is configured to determine the interface application is online and store the retrieved patient information in an online data store. At least one aspect of the client engagement and customer relationship management platform may include where the interface application is configured to determine the interface application is online and move the retrieved patient information from the offline data store to an online data store. At least one aspect of the client engagement and customer relationship management platform may include where the interface application is determined to be online when the interface application can transfer a predetermined amount of information to the client engagement and customer relationship management platform within a predetermined amount of time. At least one aspect of the client engagement and customer relationship management platform may include where the client engagement and customer relationship management platform interface resides on a device separate from a device on which the interface application resides. At least one aspect of the client engagement and customer relationship management platform may include where the messaging interface is configured to receive a message from a communication device associated with a patient of the group of patients. And, at least one aspect of the client engagement and customer relationship management platform may include where the messaging interface is configured to communicate one or more of a Short Message Service (SMS) message, a real-time message, a message including audio content, or a message including video content.

In accordance with embodiments of the present disclosure, a method is provided. The method may include receiving data at a server in a cloud network environment from various devices in a medical care facility providing health care services to at least one patient, wherein the data comprises medical data related to diagnosis and treatment of a health condition of the patient, storing the data in a core database in the cloud, extracting from the core database in the cloud, patient related information based on one or more criteria, creating a group message to be sent to a plurality of patients in accordance with the one more criteria, sending the group message to a first patient of the plurality of patients using a first communication medium, and sending the group message to a second patient of the plurality of patients using a second communication medium, wherein the first communication medium is different from the second communication medium.

Aspects of the above method may include receiving an access request for patient information residing within the core database, retrieving the patient information from the core database, and storing the retrieved patient information. Aspects may further include encrypting the retrieved patient information. Additional aspects may include receiving a message from a communication device associated with a patient of the plurality of patients. And yet, additional aspects may include a computer-readable medium comprising one or more processor executable instructions, which when executed by a processor, perform the above method.

In accordance with at least one embodiment, a method is provided. The method may include accessing, by an interface application, a core database via a client engagement and customer relationship management platform, retrieving patient information from the core database, determining if the interface application has gone offline, and storing the retrieved patient information in an offline data store located at a local storage area device of which the interface application resides.

Aspects of the above method may include determining that the interface application is online, and moving the retrieved patient information from the offline data store to an online data store located at the local storage area device of which the interface application resides. Additional aspects of the above method may include receiving a message at the interface application of the client engagement and customer relationship management platform, and providing a message via the interface application to a messaging interface of the client engagement and customer relationship management platform. Additional aspects of the above method may include providing a selection of a filter defining a group of patient records located at the core database, receiving contact information for the group of patient records, providing a group message, and sending the group message to one or more communication devices associated with each patient of the group of patients over a communication network utilizing the contact information. And yet, additional aspects may include a computer-readable medium comprising one or more processor executable instructions, which when executed by a processor, perform the above method.

The phrases “at least one,” “one or more,” and “and/or,” as used herein, are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C,” means A alone, B alone, C alone, A and B together, A and C together, B and C together, or A, B and C together.

Unless otherwise indicated, all numbers expressing quantities, dimensions, conditions, and so forth used in the specification and claims are to be understood as being modified in all instances by the term “about”.

The term “a” or “an” entity, as used herein, refers to one or more of that entity. As such, the terms “a” (or “an”), “one or more,” and “at least one” can be used interchangeably herein.

The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein.

It shall be understood that the term “means” as used herein shall be given its broadest possible interpretation in accordance with 35 U.S.C., Section 112, Paragraph 6. Accordingly, a claim incorporating the term “means” shall cover all structures, materials, or acts set forth herein, and all of the equivalents thereof. Further, the structures, materials, or acts and the equivalents thereof shall include all those described in the summary of the invention, brief description of the drawings, detailed description, abstract, and claims themselves.

The Summary of the Invention is neither intended nor should it be construed as being representative of the full extent and scope of the present invention. Moreover, references made herein to “the present invention” or aspects thereof should be understood to mean certain embodiments of the present invention and should not necessarily be construed as limiting all embodiments to a particular description. The present invention is set forth in various levels of detail in the Summary of the Invention as well as in the attached drawings and the Detailed Description and no limitation as to the scope of the present invention is intended by either the inclusion or non-inclusion of elements, components, etc. in this Summary of the Invention. Additional aspects of the present invention will become more readily apparent from the Detailed Description, particularly when taken together with the drawings.

These and other advantages will be apparent from the disclosure of the invention(s) contained herein. The above-described embodiments, objectives, and configurations are neither complete nor exhaustive. As will be appreciated, other embodiments of the invention are possible using, alone or in combination, one or more of the features set forth above or described in detail below.

### BRIEF DESCRIPTION OF THE DRAWINGS

Those of skill in the art will recognize that the following description is merely illustrative of the principles of the present invention, which may be applied in various ways to provide many different alternative embodiments. This description is made for illustrating the general principles of the teachings of this invention and is not meant to limit the inventive concepts disclosed herein.

The accompanying drawings, which are incorporated in and constitute a part of the specification, illustrate embodiments of the invention and together with the general description of the invention given above and the detailed description of the drawings given below, serve to explain the principles of the invention.

FIG. 1 depicts details of data flow between products and/or services provided by a client engagement and communication platform in accordance with embodiments of the present disclosure;

FIG. 2 depicts additional details of a client engagement and communication platform in accordance with embodiments of the present disclosure;

FIG. 3 depicts additional detail with respect to how communication is coordinated for a variety of communication mediums in accordance with embodiments of the present disclosure;

FIG. 4 depicts an example hardware configured in accordance with embodiments of the present disclosure;

FIG. 5 depicts an example architecture in accordance with embodiments of the present disclosure;

FIG. 6 depicts details of a data synchronization architecture in accordance with embodiments of the present disclosure:

FIG. 7 depicts details of a process for synchronizing information in accordance with embodiments of the present disclosure;

FIG. 8 depicts one or more organizational groups in accordance with embodiments of the present disclosure;

FIG. 9 depicts an entity relationship diagram in accordance with embodiments of the present disclosure;

FIG. 10 depicts an example interface utilized to create new appoints in accordance with embodiments of the present disclosure;

FIG. 11 depicts an example interface utilized to process a walk-in patient in accordance with embodiments of the present disclosure;

FIG. 12 depicts an example interface utilized to check out a patient in accordance with embodiments of the present disclosure;

FIG. 13 depicts an example interface utilized to send a direct message to a recipient in accordance with embodiments of the present disclosure;

FIG. 14 depicts an example interface utilized to send a group message to a plurality of recipients in accordance with embodiments of the present disclosure;

FIG. 15 depicts a flow diagram utilized to update a number of filtered recipients in accordance with embodiments of the present disclosure;

FIG. 16 depicts a process utilized to receive, process, and send messages in accordance with embodiments of the present disclosure;

FIG. 17 depicts a UML diagram in accordance with embodiments of the present disclosure; and

FIG. 18 depicts an opt-in process for opting in to SMS and marketing communications in accordance with embodiments of the present disclosure.

It should be understood that the drawings are not necessarily to scale, and various dimensions may be altered. In certain instances, details that are not necessary for an understanding of the invention or that render other details difficult to perceive may have been omitted. It should be understood, of course, that the invention is not necessarily limited to the particular embodiments illustrated herein.

### DETAILED DESCRIPTION

Although the following text sets forth a detailed description of numerous different embodiments, it should be understood that the legal scope of the description is defined by the words of the claims set forth at the end of this disclosure. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

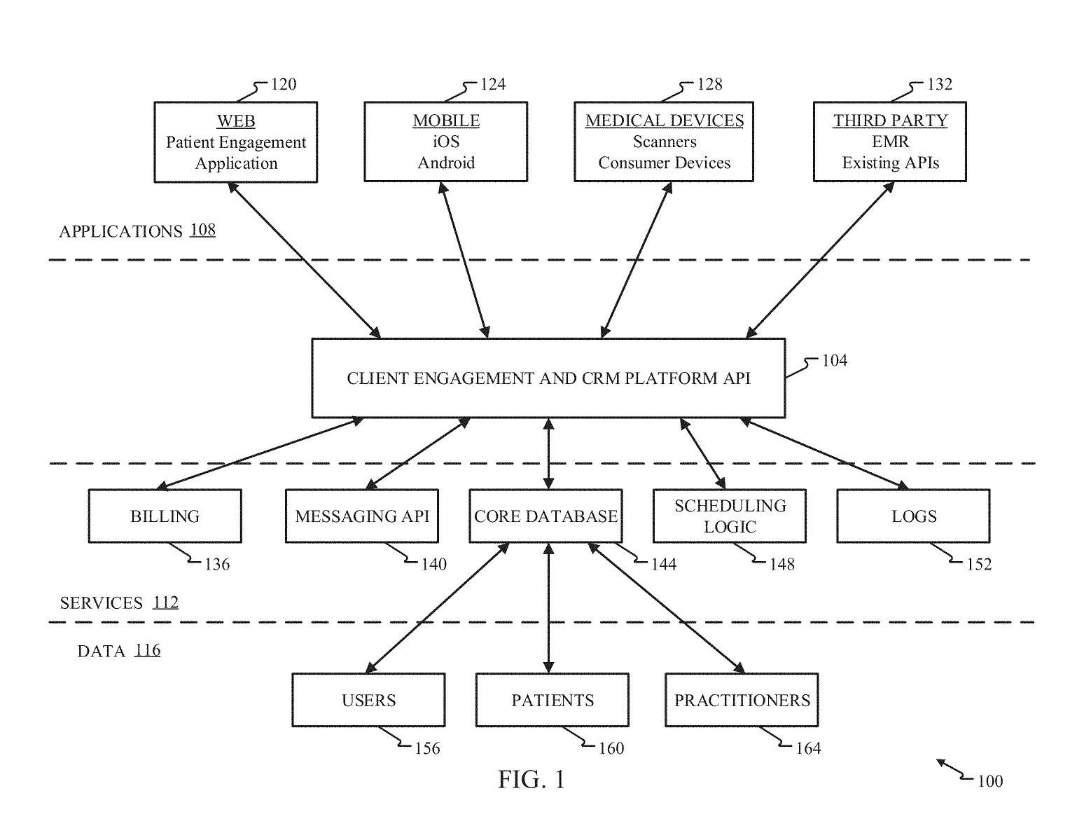

FIG. 1 depicts a hierarchical system diagram in accordance with at least one embodiment of a client engagement and customer relationship (CRM) platform 100. More specifically, FIG. 1 details the flow of data between user-facing products, such as one or more applications 108, and internal services, such as one or more services 112, offered by the client engagement and CRM platform 100. The one or more applications 108 may include, but are not limited to, web applications 120 that may be provided in one or more web browsers, mobile applications 124, medical devices 128, and other third party services 132. The one or more applications 108 may interact with the client engagement and CRM platform API 104. The one or more applications 108 may be standalone pieces of software that do not permanently persist, or store, core business data within a local hard disk. Accordingly, the one or more applications 108 may store ephemeral or semi-permanent data locally, but must be synced with core services 112 to persist data permanently. That is, for the one or more applications 108 to persist, or store, data permanently, they must communicate using the client engagement and CRM platform API 104. The client engagement and CRM platform API 104 acts as a canonical communication gateway for all pertinent business activity. That is, the client engagement and CRM platform API 104 is responsible for accepting network requests from all of the one or more applications 108 and translating the requests into pure business data and behavior, such as persisting data permanently to the core database 144. The core database 144 may be an Azure SQL database for example.

The client engagement and CRM platform API 104 may act as a gateway to all internal services 112 and data 116. That is, the core data 116, such as user-centric data 156, patient-centric data 160, and practitioner-centric data 164, may be accessed through the client engagement and CRM platform API 104. In some embodiments, the core data 116, such as user centric data 156, patient centric data 160, and practitioner centric data 164, may only be accessed through the client engagement and CRM platform API 104. The client engagement and CRM platform API 104 also handles access to the one or more services 112 and data 116. Further, the client engagement and CRM platform API 104 may handle authorization and authentication to the one or more services 112 and to access and store information to data 116. Such one or more services 112 may include, but are not limited to, billing 136, which may handle license management; a messaging API 140, which may provide users the ability to communicate with the messaging API for SMS, email, WhatsApp, etc.; a core database 144; scheduling logic 148, which may handle recurring events, such as, but not limited to, sending a recurring medication reminder; and logging and logs 152 for logging for security and HIPAA compliance. Accordingly, the architecture depicted in FIG. 1 allows any existing and future applications to utilize the client engagement and CRM platform API 104. Thus, instead of having separate services for each product and/or application 108, all products and/or applications 108 can leverage a singular interface, such as the client engagement and CRM platform API 104. Furthermore, the client engagement and CRM platform API 104 ensures consistency across products and/or applications 108, so that the same data is available to all products and/or applications 108. Importantly, this also ensures all billing and license management for all products and/or applications are managed through a single gateway, such as the client engagement and CRM platform API 104.

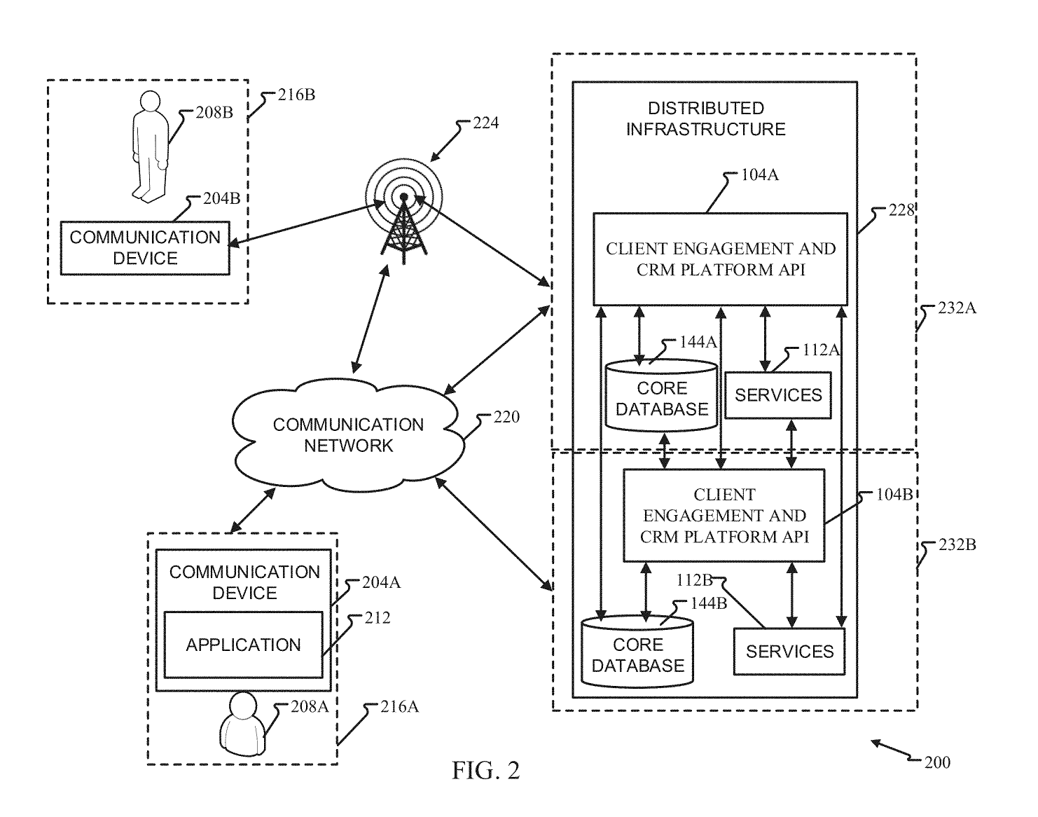

FIG. 2 depicts an example implementation 200 of the client engagement and customer relationship (CRM) platform 100 in accordance with at least one embodiment of the present disclosure. The example implementation 200 generally includes one or more users 208A-B at one or more locations 216A-B operating a communication device 204A-B to interact with the client engagement and CRM platform API 104 and other communication devices 204A-B utilizing a communication network 220. More specifically, the communication device 204A-B may comprise any type of known communication equipment or collection of communication equipment. Examples of a suitable communication device 204A-B, may include, but are not limited to, a personal computer and/or laptop with a telephony application, a cellular phone, a smart phone, a telephone, a tablet, or other device that can make or receive communications. In general, each communication device 204A-B may provide many capabilities to one or more users 208A-B who desire to interact with the client engagement and CRM platform API 104. These capabilities may include, but are not limited to, video, audio, text, applications, and/or data communications and the ability to receive a communication and/or establish a communication session with one or more users 208A-B and/or one or more services 112. For example, the communication device 204A may be a clinic workstation located at a clinic location 216A. The communication device 204A may execute or otherwise run an application 212 that includes one or more computer-executable instructions. The application 212 may comprise one or more of the applications 108. Thus, the application 212 may be a web browser 120 and may provide the user 208A with access to the client engagement and CRM platform API 104 and the services 112 associated therewith. As another example, the communication device 204B may be a video telephony device (e.g., video phone, telepresence device, a camera-equipped cellular or wireless phone, a mobile collaboration device, and a personal tablet, or laptop computer with a camera or web camera). The communication device 204B may allow the user 204B to communicate directly or indirectly with the user 208A. Such communication may be synchronous, asynchronous, and combinations thereof.

As another example, the communication device 204B may be a cellular phone having limited capability by design. For example, the communication device 204B may be able to place, establish, and receive audio communication sessions, such as telephone calls, and may be able send and receive text messages, such as SMS messages utilizing cellular, or cellular-like communication, using one or more cell towers 224. Alternatively, or in addition, the communication device 204B may be a sat phone capable of placing, establishing, and receiving audio communication sessions, such as telephone calls, and may be able send and receive text messages, such as SMS messages utilizing orbiting satellites instead of terrestrial cell sites. As another example, the communication device 204B may be a smart phone and/or tablet having limited capability due to restrictions placed on the communication device 204B, such as security restrictions, and/or due to reduced functionality because of the location, personal settings and customizations, or lack of installed apps or applications; that is, the communication device 204B, though capable of performing more advanced functions, may be limited in operation. Accordingly, the communication device 204B may utilize one of a plurality of different communication mediums and/or communication protocols when interacting with the client engagement and CRM platform API 104. The type of medium used by the communication device 204A-B to communicate with other communication devices 204A-B or the client engagement and CRM platform API 104 may depend upon the availability of communication applications, availability of bandwidth, availability and compatibility with one or more communication protocols, the location of the communication device 204A-B, and combinations thereof.

The communication network 220 may be packet-switched and/or circuit-switched. An illustrative communication network 220 includes, without limitation, a Wide Area Network (WAN), such as the Internet, a Local Area Network (LAN), a Personal Area Network (PAN), a Public Switched Telephone Network (PSTN), a Plain Old Telephone Service (POTS) network, a cellular communications network, an IP Multimedia Subsystem (IMS) network, a Voice over IP (VoIP) network, a SIP network, or combinations thereof. The Internet is an example of the communication network 220 that constitutes an Internet Protocol (IP) network including many computers, computing networks, and other communication devices located all over the world, which are connected through many telephone systems and other means. In one configuration, the communication network 220 is a public network supporting the TCP/IP suite of protocols. Communications supported by the communication network 220 include real-time, near-real-time, and non-real-time communications. For instance, the communication network 220 may support voice, video, text, web-conferencing, or any combination of media. Moreover, the communication network 220 may comprise a number of different communication media such as coaxial cable, copper cable/wire, fiber-optic cable, antennas for transmitting/receiving wireless messages, and combinations thereof. In addition, it can be appreciated that the communication network 220 need not be limited to any one network type, and instead may be comprised of a number of different networks and/or network types. It should be appreciated that the communication network 220 may be distributed. Although embodiments of the present disclosure will refer to one communication network 220, it should be appreciated that the embodiments claimed herein are not so limited. For instance, multiple communication networks 220 may be joined by many servers and networks.

Further, the client engagement and CRM platform API 104 may be distributed across one or more locations 232A-B. For instance, a distributed infrastructure 228 may be utilized by the client engagement and CRM platform API 104. The distributed infrastructure 228 may include one or more components for executing and/or running one or more programs and/or applications. That is, the distributed infrastructure 228 may include one or more servers, network components, and the like to make the client engagement and CRM platform API 104 available to one or more other devices. Such a distributed infrastructure 228 may be utilized for redundancy and disaster recovery purposes, capacity and capacity planning purposes, and/or to comply with one or more rules, regulations, or guidelines affecting how and where data may reside. For example, in instances where data 116 includes patient-centric information 160, such as medical records, requirements affecting how and where the data is stored may be different between local, regional, and national levels. Accordingly, data 116 stored in a core database 144 may need to be segmented such that the data resides within the locally, regionally, and/or nationally defined boundaries and only accessible by users within such boundaries.

As further illustrated in FIG. 2, the distributed infrastructure 228 may be distributed across a first location 232A and a second location 232B. In some instances, the client engagement and CRM platform API 104, such as client engagement and CRM platform API 104A, may reside in a single location, such as location 232A and access data 116 from core database 144A at the first location 232A and data 116 from core database 144B located a the second location 232B. Similarly, the client engagement and CRM platform API 104A may access services 112A provided from the first location 232A and services 112B provided from the second location 232B. Alternatively, or in addition, location 232B may include or otherwise comprise a client engagement and CRM platform API 104B; such client engagement and CRM platform API 104B may be in communication with the client engagement and CRM platform API 104A. That is, the client engagement and CRM platform API 104A may provide the same functionality as the client engagement and CRM platform API 104B and operate using the same or different data 116 and/or services 112. Alternatively, or in addition, the client engagement and CRM platform API 104A may provide different functionality than the client engagement and CRM platform API 104B and operate using the same or different data 116 and/or services 112. Accordingly, data 116 and/or services 112 may be segmented accordingly.

While FIG. 2 illustrates various components of the system collocated, certain components of the system can be located remotely, at distant portions of a distributed network, such as a LAN and/or the Internet, or within a dedicated system. Thus, it should be appreciated, that the components of the system can be combined into one or more devices, such as a server, or collocated on a particular node of a distributed network, such as an analog and/or digital telecommunications network, a packet-switch network, or a circuit-switched network. It will be appreciated from the preceding description, and for reasons of computational efficiency, that the components of the system can be arranged at any location within a distributed network of components without affecting the operation of the system. For example, the various components can be located in one or more communications devices, at one or more users' premises, or some combination thereof. Similarly, one or more functional portions of the system could be distributed between a telecommunications device(s) and an associated computing device.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire and fiber optics, and may take the form of acoustic or light waves, such as those generated during radio-wave and infra-red data communications.

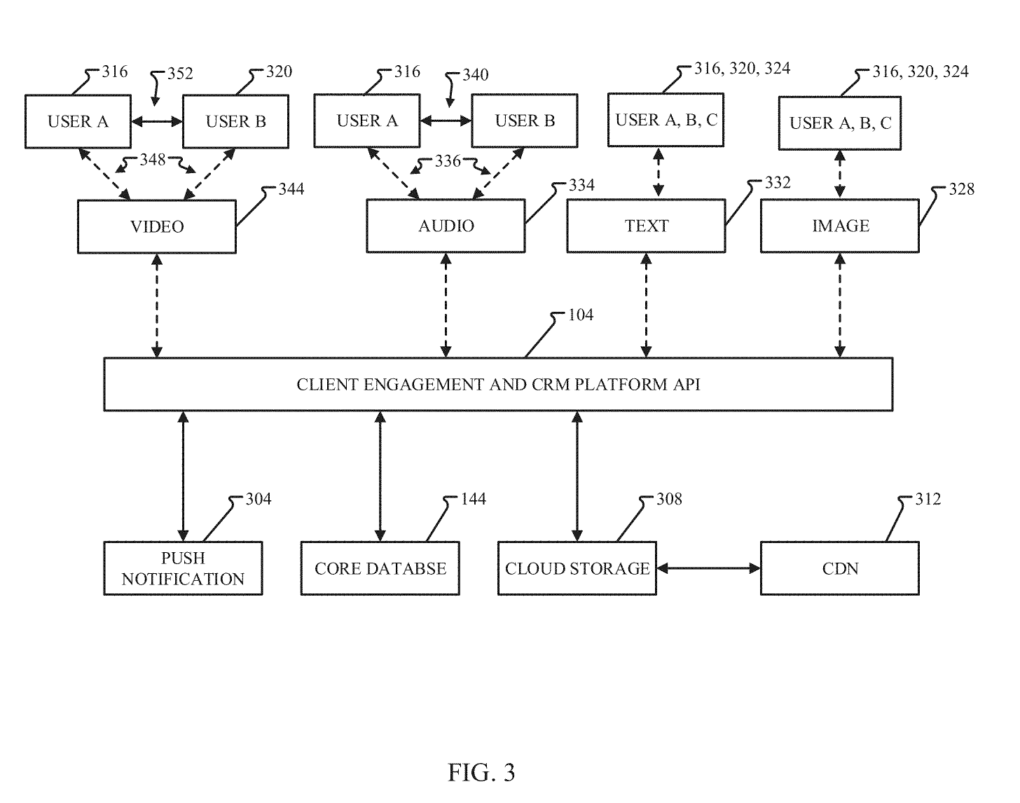

FIG. 3 details one or more communication mediums that may be utilized by users 316 and 320 to interact with one another and/or to interact with the client engagement and CRM platform API 104. It should be understood that user 316 may be the same as user 208A-B; further, though depicted as user 316, the communication mediums available to user 316 are understood to be those communication mediums available to and supported by one or more communication devices 204A-B. It should further be understood that user 320 may be the same as user 208A-B; further, though depicted as user 320, the communication mediums available to user 320 are understood to be those communication mediums available to and supported by one or more communication devices 204A-B. It should additionally be understood that user 324 may be the same as user 208A-B; further, though depicted as user 324, the communication mediums available to user 324 are understood to be those communication mediums available to and supported by one or more communication devices 204A-B.

Text-based communication 332 can be initiated by any authorized party, such as User A 316, User B, 320, and User C 324, in the system. The written text is transmitted over the client engagement and CRM platform API 104 and persisted into the core database 144. In the event that a user needs to be notified about the transmitted text, a notification can be sent to the user's device via push notification 304, for example.

Image-based communication 328 can be initiated by any authorized user, such as User A 316, User B 320, and User C 324, in the system. For example, an image of a hand-written form scanned by a front desk worker 324 at a clinic may be attached to a patient record. The image is transmitted over the client engagement and CRM platform API 104, and metadata may be stored for later use in the core database 144 and stored permanently in cloud-based storage, such as cloud-based storage 308. The cloud storage 308 can then geographically distribute the image on a content delivery network (CDN) 312 to improve download speeds across the world.

Audio-based communication 334 can be initiated by any authorized party in the system. For example, a patient may speak directly with a practitioner. In such a case, User A 316 indicates they would like to communicate with another user, User B 320. This communication intention is transmitted to the client engagement and CRM platform API 104 to ensure both parties would like to connect, and User B 320 is notified of the intention. Coordination information 336 is passed to both parties and these parties can connect directly 340 (via WebRTC, for example) without any need or burden from the client engagement and CRM platform API 104. Upon finishing the audio-based communication 334, the client engagement and CRM platform API 104 is notified of the closure. Further, such communication may be recorded in some instances.

Video-based communication 344 can be initiated by any authorized party in the system. For example, a patient may directly video conference with a practitioner. In such a case, User A 316 indicates they would like to communicate with another user, User B 320. This intention is transmitted to the client engagement and CRM platform API 104 to ensure both parties would like to connect, and User B 320 is notified of the intention. Coordination information 348 is passed to both parties and these parties can connect directly 352 (via WebRTC, for example) without any need or burden from the client engagement and CRM platform API 104. Upon finishing the video-based communication 344, the client engagement and CRM platform API 104 is notified of the closure.

Additional methods of communication are further contemplated herein. For example, users 316, 230, 324 may communicate using various forms of synchronous communication, asynchronous communication, and combinations thereof. Further protocols are contemplated as well. Further, e-mail may be one form of communication. Further still, in some instances, a dedicated application, such as one or more of the applications 108 may be involved in the communication process. In some embodiments, one or more cross-messaging platforms may be used. An example of a cross-messaging platform includes, but is not limited to, Skype, Facebook Messenger, and/or WhatsApp. Further, User A 316 may initiate a communication session with User B 320 and communicate using such a communication utilizing one or more methods described in European Patent Application No. EP20130425126, which is incorporated herein by reference in its entirety for all that it teaches and for all purposes. Alternatively, or in addition, User A 316 may initiate a communication session with User B 320 and communicate using such a communication utilizing one or more methods described in U.S. Patent Application No. 2015/0040029, which is incorporated herein by reference in its entirety for all that it teaches and for all purposes. Alternatively, or in addition, one or more communication sessions may be recorded utilizing one or more methods described in U.S. Patent Publication No. 2014/0270104, which is incorporated herein by reference in its entirety for all that it teaches and for all purposes.

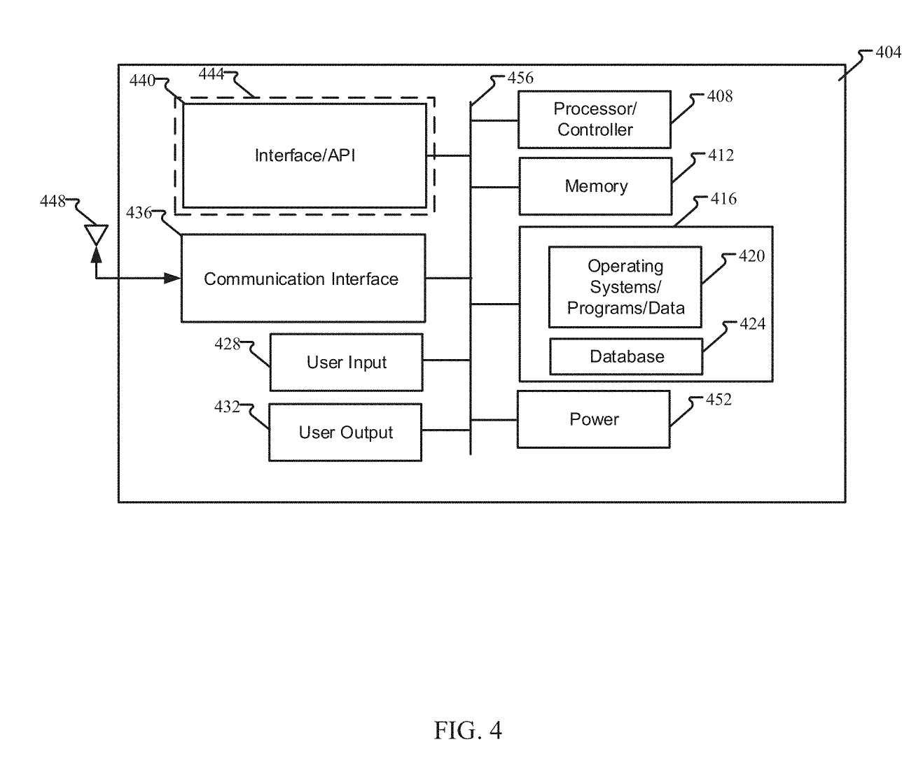

FIG. 4 depicts additional details of one or more systems 404 in accordance with embodiments of the present disclosure. The communication device 204A-B may comprise the system 404; alternatively, or in addition, the communication device 204A-B may include one or more components of the system 404. That is, one or more components of the system 404 may be utilized such that one or more computer-executable instructions of the communication device 204A-B are executed by the system 404. Alternatively, or in addition, the distributed infrastructure 228 may comprise the system 404; alternatively, or in addition, the distributed infrastructure 228 may include one or more components of the system 404. That is, one or more components of the system 404 may be utilized such that one or more computer-executable instructions of the distributed infrastructure 228 are executed by the system 404. It should be understood that different systems 404 may be used for each of the communication devices 204A-B, the distributed infrastructure 228, and/or hardware for use with the client engagement and CRM platform API 104, the one or more services 112, the one or more applications 108, and/or one or more functions associated with the data 116.

The system 404 may include a processor/controller 408 capable of executing program instructions. The processor/controller 408 may include any general-purpose programmable processor or controller for executing application programming. Alternatively, or in addition, the processor/controller 408 may comprise an application specific integrated circuit (ASIC). The processor/controller 408 generally functions to execute programming code that implements various functions performed by the system 404. The processor/controller 408 of one or more of the systems 404 may operate to execute one or more computer-executable instructions of the client engagement and CRM platform API 104A as will be described herein. Alternatively, or in addition, the processor/controller 408 of one or more of the systems 404 may operate to execute one or more computer-executable instructions of one or more services 112 and/or one or more functions associated with the data 116. Alternatively, or in addition, the processor/controller 408 of one or more of the systems 404 may operate to execute one or more computer-executable instructions of the communication devices 204A-B, as will be described herein.

The system 404 additionally includes memory 412. The memory 412 may be used in connection with the execution of programming instructions by the processor/controller 408, and for the temporary or long-term storage of data and/or program instructions. For example, the processor/controller 408, in conjunction with the memory 412 of the system 404, may implement one or more modules, web services, APIs and other functionality that is needed and accessed by a communication device, such as communication device 204A-B. The memory 412 of the system 404 may comprise solid-state memory that is resident, removable, and/or remote in nature, such as DRAM and SDRAM. Moreover, the memory 412 may include a plurality of discrete components of different types and/or a plurality of logical partitions. In accordance with still other embodiments, the memory comprises a non-transitory computer-readable storage medium. Such a medium may take many forms, including, but not limited to, non-volatile media, volatile media, and transmission media.

The system 404 may include storage 416 for storing an operating system, one or more programs, and additional data 420. The storage 416 may be the same as or different from the memory 412. For example, the storage 416 of the system 404 may include a database 424 for storing data 116. Of course, the database 424 may be distributed across one or more systems 404. Further, the database 424 may be the same as or similar to the core database 144.

In addition, user input devices 428 and user output devices 432 may be provided and used in connection with the system 404. For example, a first user may enter information, or initiate a communication with the system 404, by directing a web browser to a website served and/or provided by the system 404 by entering a website address or by clicking on a hyperlink associated with the website. Alternatively, or in addition, a user may enter information, or initiate a communication with the system 404, by interacting with a user interface of the system 404. Examples of user input devices 428 include a keyboard, a numeric keypad, a touch screen, a microphone, scanner, and pointing device combined with a screen or other position encoder. Examples of user output devices 432 include a display, a touch screen display, a speaker, and a printer. Further, user output devices 432 may provide one or more interfaces for user interfacing.

The system 404 generally includes a communication interface 436 to allow for communication between communication devices, such as communication devices 204A-B, and the system 404. The communication interface 436 may support 3G, 4G, cellular, WiFi, Bluetooth®, NFC, RS232, RF, Ethernet, one or more communication protocols, and the like. In some instances, the communication interface 436 may be connected with an antenna 448. Alternatively, or in addition, the communication interface 436 may be connected to one or more mediums for accessing the communication network 220.

The system 404 may include an interface/API 440. Such interface/API 440 may include the necessary functionality to implement the client engagement and CRM platform 104 or a portion thereof. Alternatively, or in addition, the interface/API 440 may include the necessary functionality to implement one or more services 112 and/or one or more functions of the data 116. In instances where the system 404 comprises one or more communication device 204A-B, the interface/API 440 may include the necessary functionality to implement one or more of the applications 108. It should be appreciated that the interface/API 440 may be distributed across one or more systems 404, such that the interface/API 440 represents a portion 444 of an interface/API. Communications between various components of the system 404 may be carried by one or more buses 456. Moreover, power 452 can be supplied to the components of the system 404. The power 452 may, for example, include a battery, an AC to DC converter, power control logic, and/or ports for interconnecting the system 404 to an external source of power.

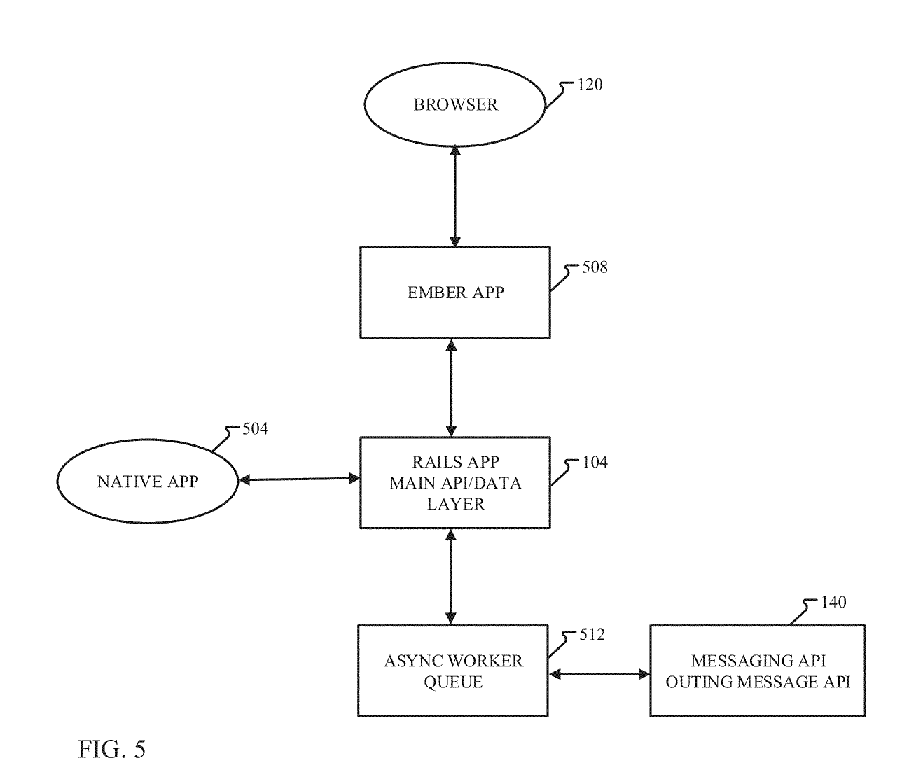

FIG. 5 provides additional details of the patient engagement and CRM platform system 100 in accordance with embodiments of the present disclosure. The client engagement and CRM platform API 104 may be built with Ruby on Rails and Ember.js. Ruby on Rails may run the client engagement and CRM platform API 104 layer that the Ember application 508 communicates with for data. A bulk of the business logic is within the Ember application that runs in a browser, for example. The Rails application manages authentication, authorization, and data persistence. The Ember application 508 renders data and presents all interfaces for the end-user.

More specifically, a browser, such as web browser 120 may interact with or otherwise utilize application-specific functionality implemented via one or more frameworks. For example, an Ember framework may be provided by the patient engagement and CRM platform system 100 such that application-specific functionality is pushed to one or more devices 204A-B. Hardware, such as a system 404, may serve application-specific information to the one or more communication devices 204A-B via an Ember framework. More information about Ember can be found in the document entitled “Ember.js Guides,” created by Precious Jahlom Agboado, published on 2014 Oct. 4, which is incorporated herein by reference in its entirety for all that it teaches and for all purposes. Alternatively, or in addition, a native app 504, or native application may be implemented in one or more of the applications 108 and/or one or more of the communication devices 204A-B. That is, a user, such as user 208A-B, may interact with or otherwise access the client engagement and CRM platform API 104 via a native app 504.

The Ember App 508 may interact with the client engagement and CRM platform API 104. The client engagement and CRM platform API 104 may utilize one or more frameworks, such as a Rails framework. More information about Ember can be found in Hartl, Michael. Ruby On Rails Tutorial: Learn Web Development with Rails. third ed. Addison-Wesley Professional Ruby Series. New York: Addison-Wesley, 2015, which is incorporated herein by reference in its entirety for all that it teaches and for all purposes. Further, in one or more implementations of the client engagement and CRM platform API 104, one or more asynchronous jobs may be implemented. Importantly, one or more asynchronous jobs may include one or more messages to be processed by a message API, such as messaging API 140. Additional details of the messaging processes, flows, and message creation is described herein. Such asynchronous jobs may be implemented utilizing one or more asynchronous worker queues 512; the asynchronous worker queue 512 may be implemented in a Sidekiq worker queue for example. A message may be sent via an outing message portion of a messaging API 140.

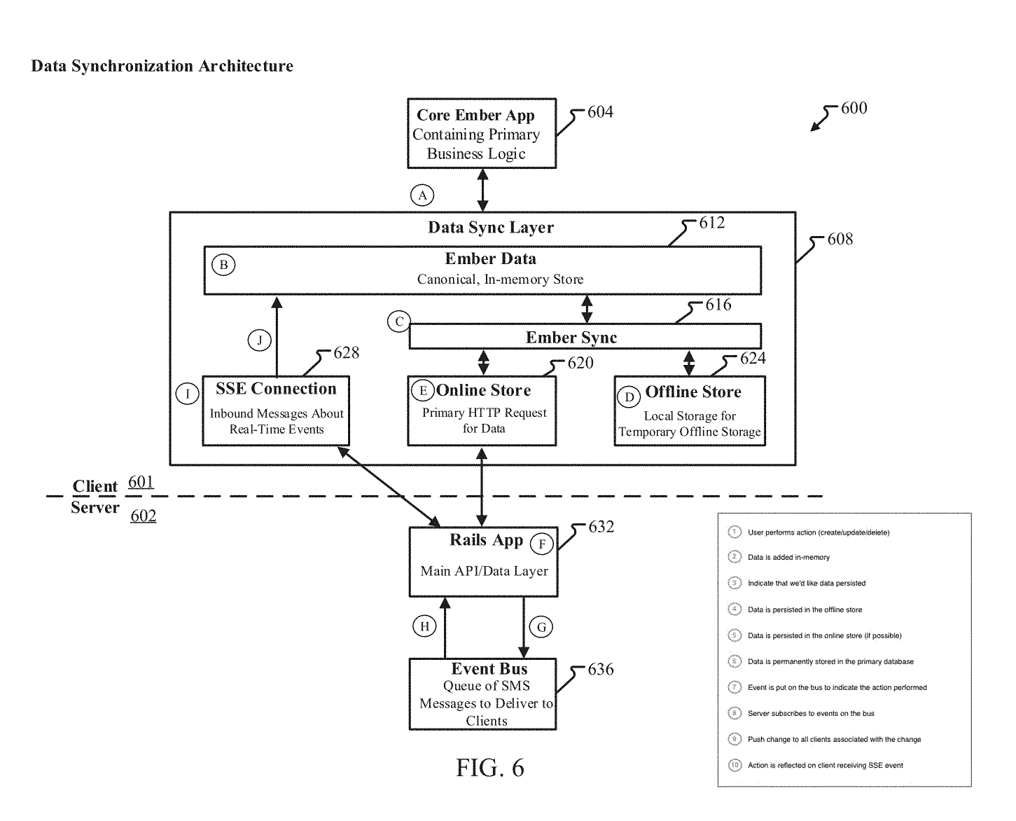

FIG. 6 provides additional details with respect to a synchronization architecture 600 that may be implemented within the patient engagement and CRM platform system 100. That is, the patient engagement and CRM platform 100 is capable of functioning offline. Intermittent and failed connections are common in Africa and other geographic regions, but the one or more applications 108 are fully functional while working offline. Patients, practitioners, appointments, messages, and everything else can be created, modified, and deleted while in an offline state. The data will then be synced with the server when the user regains their internet connection. For example, a user, such as user 208A-B, may interact with the Ember app 508 utilizing a web browser 120 as previously described. Data provided by the user 208A-B may be added into memory of a communication device, such as communication device 204A-B. Based on an indication that such data provided to the memory of the communication device 204A-B should persist, the data is first provided to an offline store where such data is temporally stored. If a connection between the communication device 204A-B and the patient engagement and CRM platform API has been established or is to be established, the data is then provided to an online store where such data is sent, via hyper text transfer protocol for example, to the core database 144 utilizing the patient engagement and CRM platform API 104. Further, an indication of such data storage event may be provided to the user, such as user 208A-B, via an event bus, where the user receives a notification, such as a push notification of such storage event. As depicted in FIG. 6, the data synchronization process 600 may be split between the client 601 and a server 602 and may follow a client/server model. As can be appreciated, the client 601 and the server 602 can be same as or similar to the clients and servers previously discussed; accordingly, details are omitted for purposes of clarity. As previously discussed, the client (e.g. 601) and the server (e.g. 602) may each be implemented utilizing different systems 404.

As depicted in the data synchronization process 600, data may initially be provided to a core ember app at step 604; the core ember app may include the primary business logic utilized to facilitate data transfer and storage. For example, the core ember app may include one or more rules dictating when and how data is moved within the data synchronization layer. Accordingly, the data from the core ember app may be proved to the data sync layer at step 608. Within the data sync layer process 608, multiple sub processes may run to store and maintain data synchronization. That is, the ember data may be received and stored within the memory at step 612 as previously discussed. Depending on a state of the client 601, for example, whether client 601 is online or offline, the data may move to be synchronized with the server 602 at step 616. That is, the ember data may be synchronized with the server 602 using one or more of a data transfer mechanism. At least one example of a data transfer mechanism may include, but is not limited to, an HTTP request to move and/or synchronize data at step 620. In that the client 601 has the ability to work in both an offline mode (e.g. connectionless mode) or in an online mode (connected mode), the app improves the operation of the client 601 itself. Moreover, there may be many ways to determine if the client 601 is offline. For example, such a determination may be based on how much data can be transmitted to or from the server 602 (e.g., the interface) in a predetermined amount of time. Moreover, a simple try and connect method may be employed, where the client 601 attempts to establish a connection with the server 602, if after a certain period of time, no response or an inadequate response is received, the connection state may be determined to be offline. As another example, a communication tool such as PING may be used.

The data may then be moved to a data layer within the server 602 at step 632. If the client 601 is offline, the data may be stored locally and temporarily at step 624 in an offline data storage area. Once the data is moved to the server 602, the data may be processed and/or added to an event bus at step 636 such that messages from the client 601 may be handled at server 602 at step 636.

In instances where the server 602 provides information to the client 601, the data synchronization process 600 may be reversed. That is, in some instances, data on an even bus may be provided to a main data layer at step 636. The main data layer may determine whether data is to be provided to the client 601 via a server side event (SSE) at step 628, or by another process at step 620, where such data is stored, synched, and provided to the core ember app at step 604.

As another example, at step A, a user may perform an action such as creating, updating, editing, or other operation on some form of data, such as profile information, appointment information, etc. At step B, the data may be added into memory, where if data is indicated that it should be persistent data (e.g. persist) at step C, the data is stored in an offline store at step D or stored in an online store at step E. At step F, the data is stored in a permanent database, where such event may be put on the bus to indicate that the action, e.g. data storage action, was performed. At step H, the server may subscribe to an event on the bust, push a change to one or more clients at I, and reflect such change at the clients by having the clients receive the SSE event at J.

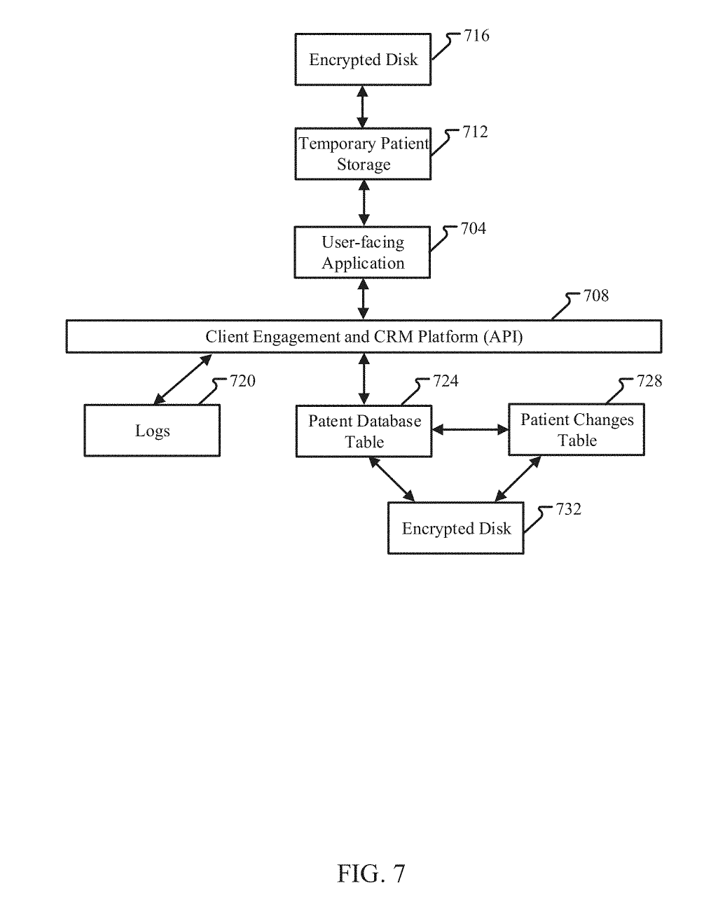

FIG. 7 provides additional details with respect to an implementation of data stored in accordance with embodiments of the present disclosure. In some instances the flow of FIG. 7 may coincide with the steps taken to be in compliance with HIPAA. The primary actions taken involve appropriate encryption and logging. End-users, such as users 208A-B, are presented with an application, such as one or more applications 108, that allows them to view or manage patient information, such as patient data 160. Patient data 160 may be created locally within the application 108 or pulled from the client engagement and CRM platform API 104 (708). When the application 704, such as one or more applications 108, opts to store data locally in a semi-permanent fashion (e.g., because the application is offline), the application 704 can choose to persist the patient data locally at step 712. The local patient storage is encrypted on disk at step 716, such as in the data 416 with the user's password as the encryption key (or, any other key known only to the user). For the client, this meets the encryption at rest requirements of HIPAA.

When the data is transmitted to the client engagement and CRM platform API 104 (e.g. 708) for long term, permanent storage, the client engagement and CRM platform API 104 (708) immediately logs the request at step 720, tracking details about the patient that was affected and the user making the request. This meets the PHI diagnostic tracking requirements of HIPAA. Patient information is then permanently persisted to the patient database table 160 at step 724 in the core database 144. The actual changes that were made are tracked in the patient changes table at step 728. This includes who made the change, what fields were changed, and at what time. Both of these tables, as well as the remainder of the core database 144, are encrypted on disk at step 732. For a server storing and/or providing such information, this meets the encryption at rest requirements of HIPAA.

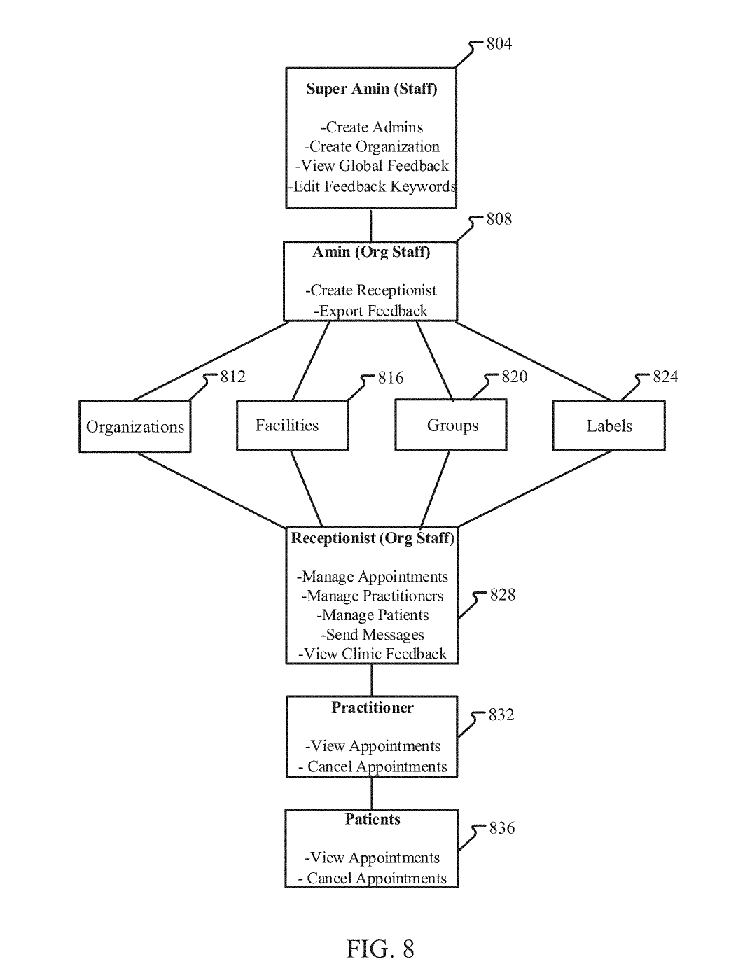

FIG. 8 provides details in accordance with one or more user roles associated with the client engagement and CRM platform API 104. That is, “role based access,” constrain a user in their access to system functionality and data. The authorization of these roles is controlled by the client engagement and CRM platform API 104. For example, a super admin 804 may create other admins, create additional organizations, view and edit global feedback. Within the context of a clinic, an admin 808 at the clinic for example, may create user roles that include receptionists. The admin 808 may further create roles associated with the organization 812, facility 816, group 820, and/or additional labels 824. Again, within the context of a clinic, a receptionist 828 may manage appointments, manage practitioners, manage patients, manage groups, send messages, and view feedback associated with the clinic. Practitioners 832 may view their appointments and cancel appointments. That is, this role grants access to an individual practitioner's appointment schedule. It allows them to change their own account information, but they cannot create patients, appointments or messages. Patents 836 can view their appointments and cancel their appointments as well. That is, the patient is able to see their individual appointments. They can see past and upcoming appointments and the details of those appointments. They can access their own health data.

Other roles may include, but are not limited to the following:

Staff—Staff users can create new organizations and user accounts. An organization represents a business entity or customer. A staff user can create any type of user account.

Owner—The owner role pertains to a specific organization. The owner can perform all actions within the system for their organization. They can change details about the organization, create sub-organizations to separate data access, and manage users accounts for the organization. Front Desk Worker—The front desk worker intakes patients and manages appointments. This role does not have access to business data, payment data, or patient health data. They can send messages to a group of patients or contact patients or practitioners directly.

Health Worker—The health worker manages patient health data. This role cannot send group messages or see business and payment data.

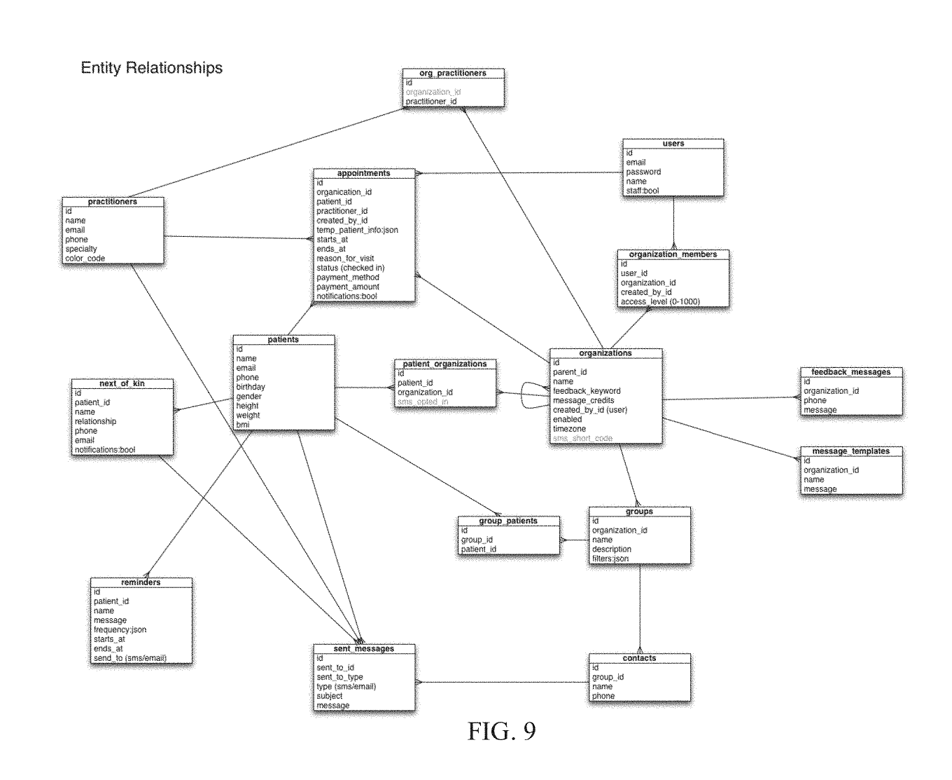

FIG. 9 illustrates one or more entity relationships that may be utilized in the patient engagement and CRM platform system 100 and the patient engagement and CRM platform API 104. More specifically, the entity relationships depicted in FIG. 9 may be utilized when applying filters to send messages to one or more selected patients or parties. Additionally, the entity relationship depicted in FIG. 9 may be utilized to access and/or store patient-centric data 160, user-centric data 156, and practitioner-centric data 164.

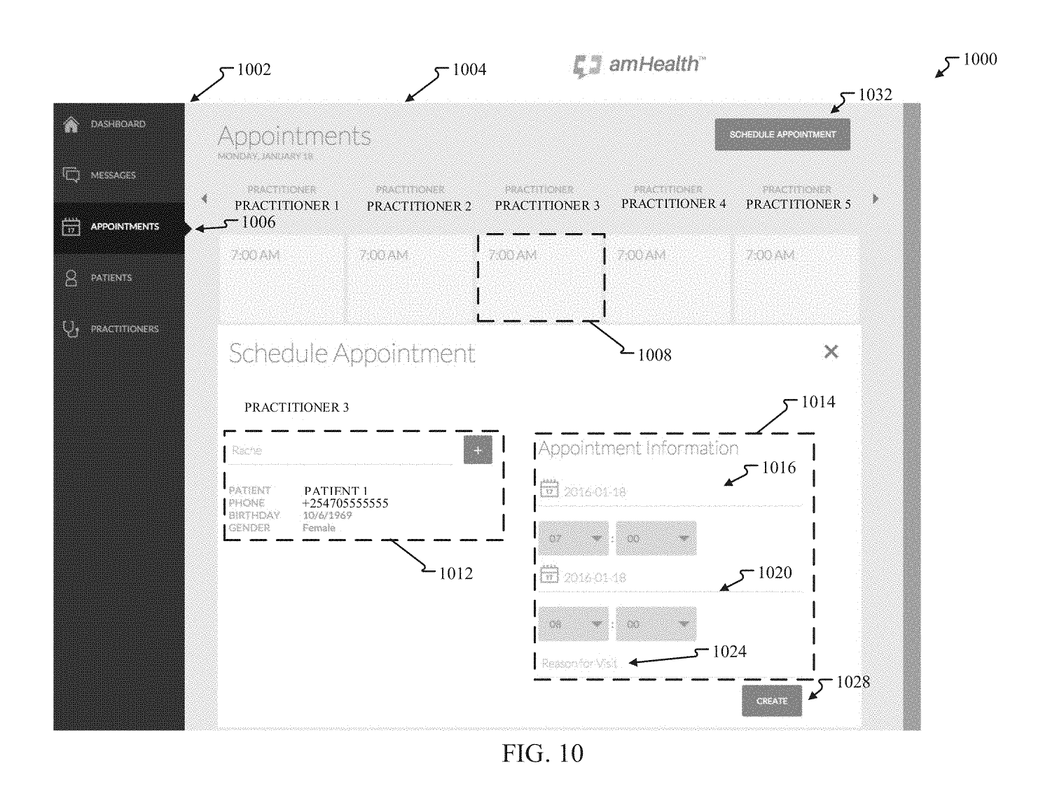

FIG. 10 depicts an example interface 1000 and process that may be utilized to create one or more appointments utilizing the patient engagement and CRM platform API 104. More particularly, the interface 1000 may include a dashboard 1002, and a main information window 1004. The interface 1000 may be utilized when a patient would like to schedule an appointment in the future (as opposed to walking in), the patient may call the facility, such as a clinic 216A and talk to a front desk worker (FDW), such as a user 208A. The FDW navigates to the appointments page 1000 by selecting an icon 1006 on the dashboard 1002. The FDW may select the date for which they would like to see appointments. As one example, a calendar may be accessible allowing the FDW to find and select a future date. The FDW finds an open appointment block and clicks the block 1008 to begin scheduling. The FDW can find an existing patient in the system or create a new patient utilizing the patient information fields 1012. For example, as depicted in FIG. 10, the FDW found an existing patient and the existing patient's information is displayed in the patient information fields 1012. The FDW then fills in appointment related information into the appointment related information fields 1014. The appointment related information fields 1014 may includes an appointment start time 1016, an appointment end time 1020, and a reason for the visit 1024. The FDW may then create the appointment by selecting the create button 1028. The new appointment will then show up in the block 1008 the FDW originally selected. If the FDW would like to create the appointment without selecting the date beforehand (since the visual schedule is only for one day), the FDW may also schedule the appointment through the “Schedule Appointment” button 1032.

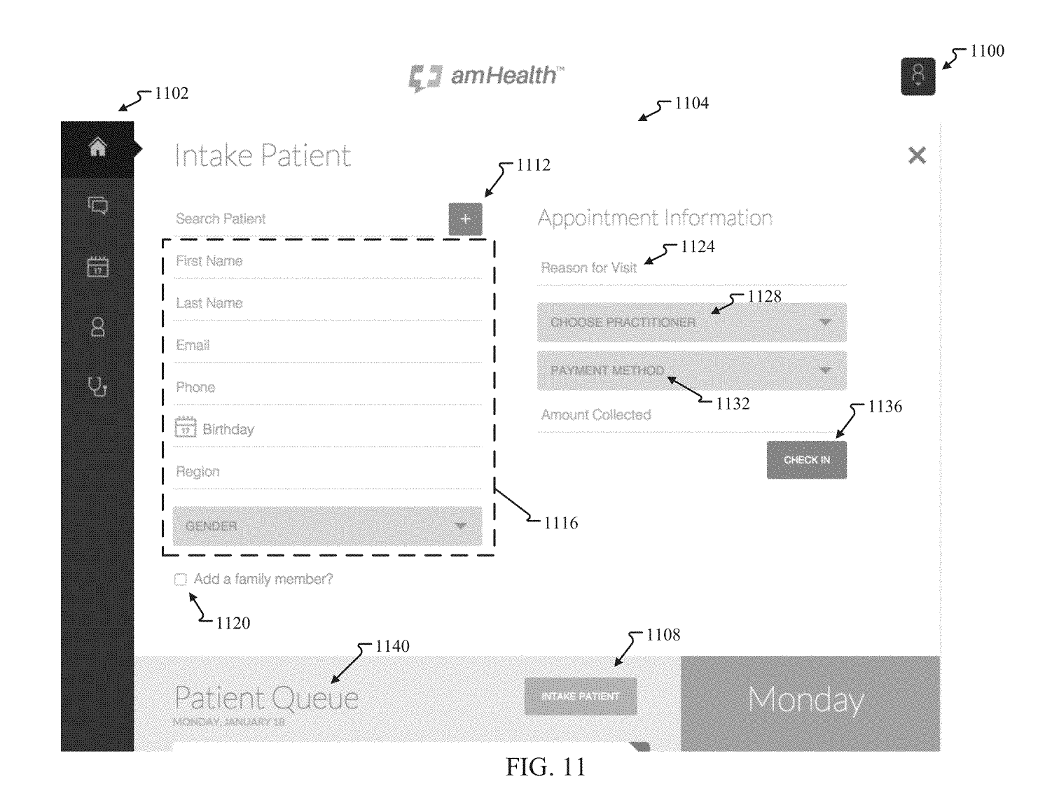

FIG. 11 depicts an example interface 1100 and process that may be utilized to check-in a patient, such as a walk-in patient, utilizing the patient engagement and CRM platform API 104. More particularly, the interface 1100 may include a dashboard 1102, and a main information window 1104. The interface 1100 may be utilized when a patient walks into a facility, such as a clinic 216A, without an appointment and visits a front desk worker (FDW), such as a user 208A, and indicates that they would like to see a practitioner. The FDW initiates the walk-in process by visiting the dashboard 1102 and selecting the “Intake Patient” icon 1108. This interface 1100 presents a form, or window area 1112, where the FDW can either search for a patient or add a new patient to the system. As depicted in FIG. 11, the FDW adds a new patient. Accordingly, the FDW fills out basic patient information 1116 and asks if the patient would like to add a family member to the system. If the patient would like to add a family member to the system, the FDW may the select the “add family member” checkbox and an association may be created between the user and the family member; such association may be reflected in the entity relationship as depicted in FIG. 9. Accordingly, the FDW may fill out the family member's name, email, phone number and relationship to the patient. The FDW then supplies the reason for visit in the field 1124 and selects or assigns the patient to a practitioner that the patient will be seeing using the field 1128. In some instances, the available practitioners that may be available to be assigned may be filtered according to one or more criteria.

Optionally, the FDW is able to select the payment method and amount if applicable utilizing the fields 1132. The FDW then selects the “Check In” button 1136 and the patient is added to the system and their appointment is placed within the Patient Queue 1140.

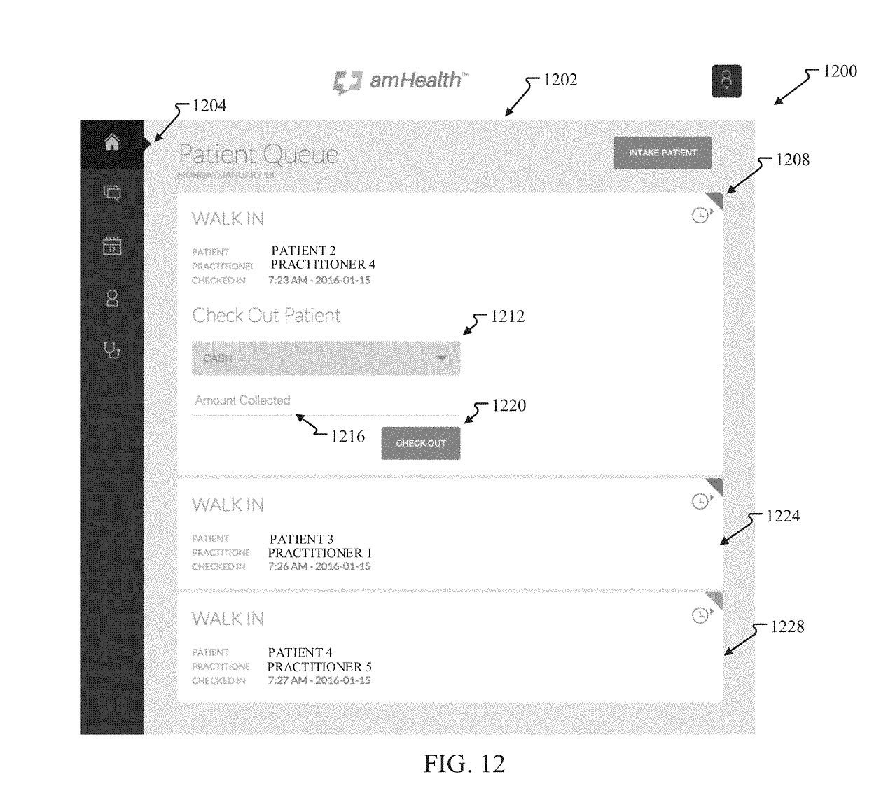

FIG. 12 depicts an example interface 1200 and process that may be utilized to check-out a patient utilizing the patient engagement and CRM platform API 104. More particularly, the interface 1200 may include a dashboard 1204, and a main information window 1202. Once a patient has entered the facility, such as a clinic 216A, the patient is “check-in.” After checking in, the patient may visit a practitioner. Before leaving the facility, the patient must “check-out.” The interface 1200 may be utilized to checkout such patient. For example, the patient visits the front desk worker (FDW), such as user 208A, before leaving the facility, such as the clinic 216A. The FDW navigates to the dashboard 1204. The FDW then asks the patient for their name, and finds them on the list, or queue, of checked in patients. Within the patient queue, there may be multiple “checked-in” patients 1214 and 1228. Accordingly, the FDW expands the “check out” button 1208. The FDW fills out the payment method and payment amount collected in the fields 1212 and 1216 respectively, and selects the “check out” button 1220. Upon being checked out, the appointment is removed from the dashboard and the patient is free to leave the facility.

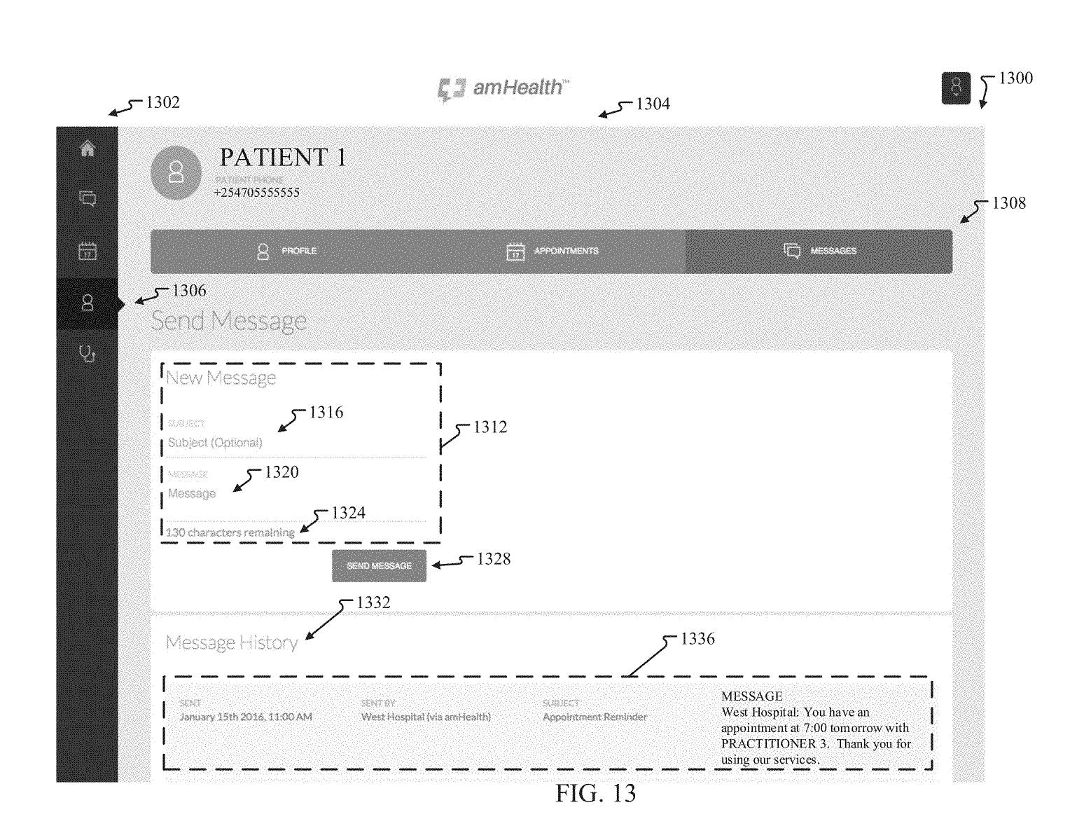

FIG. 13 depicts an example interface 1300 and process that may be utilized to send a direct message to a patient utilizing the patient engagement and CRM platform API 104. More particularly, the interface 1300 may include a dashboard 1302, and a main information window 1304. In the event that a front desk worker (FDW), such as the user 208A, needs to contact a patient directly, the FDW may send the patient a message that will be received by the patient. The message may be sent and received using, for example SMS, email, and/or phone call. Further, WhatsApp and/or other cross-platform messaging platforms may be utilized to send and/or receive such a direct message. Accordingly, the FDW navigates to the patient profile by selecting a patients page 1306 from the dashboard 1304, and then navigating to the patient's profile. The FDW navigates to the patient's messages page 1308. The FDW optionally fills out the message details 1312, a subject 1316, and a message body 1320. As the FDW fills out the message body 1320, the count 1324 of the total allowed characters decrements. Upon finishing the message, the FDW selects the “Send Message” button 1328 to finish the message. The newly created message shows up on the top of the patient's “Message History” 1332. Other messages, such as message 1336, may be accessible and viewable by the FDW as well. That is, the message history 1332 logs each message sent and/or received from the patient.

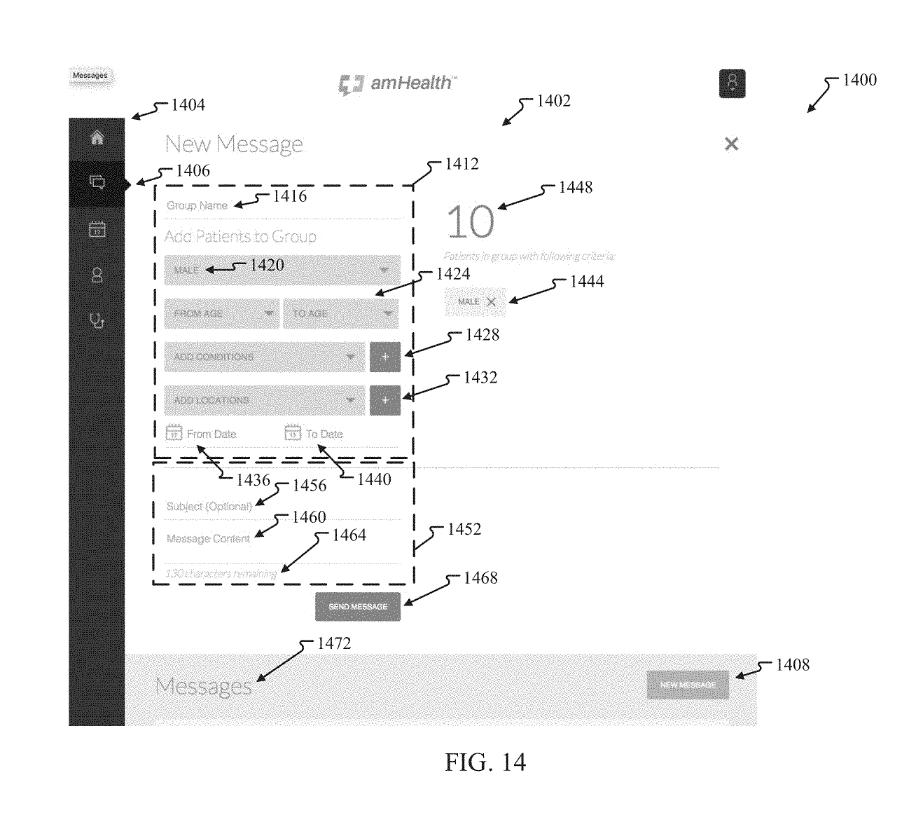

FIG. 14 depicts an example interface 1400 and process that may be utilized to send a message to a group of patients utilizing the patient engagement and CRM platform API 104. More particularly, the interface 1400 may include a dashboard 1404, and a main information window 1402. A front desk worker (FDW), such as user 208A, may need to send a group message to a plurality of patients, and in some instances, non-patients. For example, the FDW may uncover new information about a condition that should be conveyed to patients suffering from that condition. As another example, an FDW may send a message to family members associated with a patient. The FDW navigates to the group messages page 1406. The FDW may select the “New Message” button 1408 to indicate the FDW would like to start creating a new group message. The FDW then fills out the group details 1412. The group details may include the name of the group 1416. The FDW may additionally specify filters for the group of patients. For example, the FDW may fill out a gender 1420 filter, an age range 1424 filter, one or more health conditions 1428 filters, one or more locations 1432 filters, a start date 136 of when the patient was added to the system, and end date 1440 of when the patient was added to the system. Upon selecting a filter, the filter is added to the list of applied filters 1440 and the number 1448 of total patients in the system that match those filters is updated. Group message details 1452 may also be added. The group message details 1452 may include, but are not limited to the subject 1456 of the group message, and the message content or body 1456. As the FDW fills out the message body 1460, the count 1464 of the total allowed characters decrements. Upon finishing the message, the FDW selects the “Send Message” button 1468 to finish the message and create the group. The newly created message shows up under the list of sent messages 1472 to the group. The group with the applied filters is saved for later so the message can be reset to the same group.

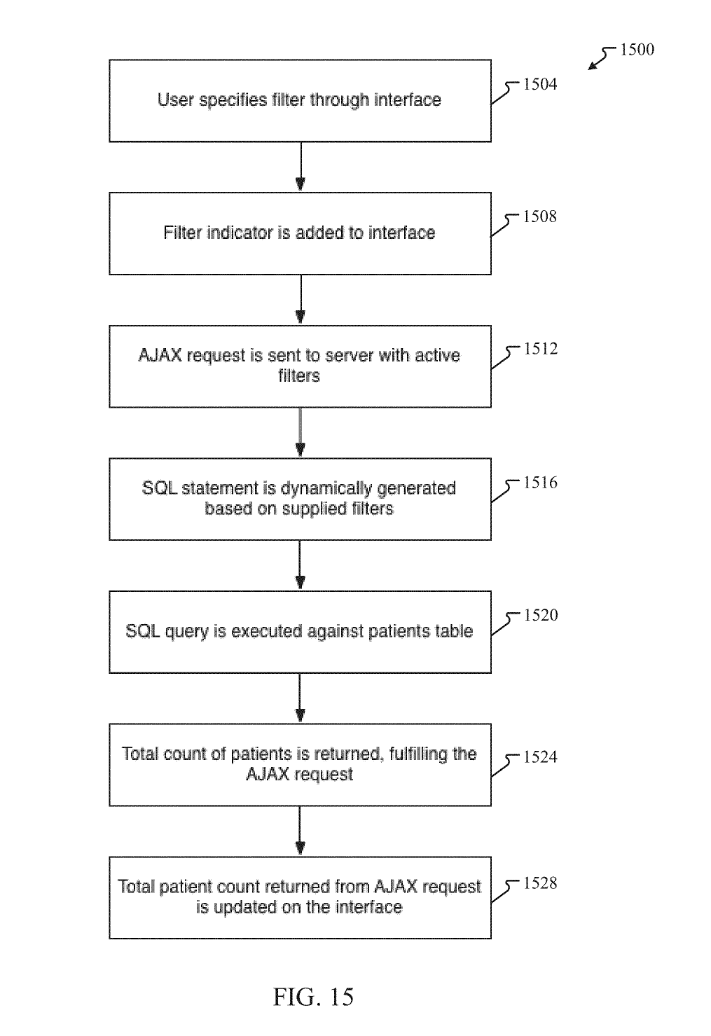

Referring now to FIG. 15, a method 1500 is discussed in accordance with embodiments of the present disclosure. Method 1500 describes how the total patient count 1448 updates as a front desk worker (FDW) selects group filters. The patient count 1448 indicates to the FDW how many patients will be sent a message and an approximate number total message credits they will be using to send the group message. Method 1500 is in embodiments, performed by a device, such as the system 404. More specifically, one or more hardware and software components may be involved in performing method 1500. In one embodiment, the patient engagement and CRM platform API 104 may be involved in performing one or more of the steps of method 1500. The method 1500 may be executed as a set of computer-executable instructions executed by a computer system and encoded or stored on a computer-readable medium. Hereinafter, the method 1500 shall be explained with reference to systems, components, modules, software, etc. described with FIGS. 1-14.

Method 1500 may continuously flow in a loop, flow according to a timed event, or flow according to a change in an operating or status parameter. For example, method 1500 may be initiated at step 1504 where a user may specify a filter through an interface, such as interface 1400. When the FDW selects a group filter at step 1504, the Ember application, such as ember app 508, dynamically recognizes a new filter has been applied. At step 1508, the filter is then added to the interface 1400 to indicate that the filter has been applied. The Ember application 508 makes a request to a server, such as system 404, via the patient engagement and CRM platform API 104 to get the canonical number of patients in the system for that organization at step 1512. Based on the list of filters sent to the server, the server dynamically generates an SQL statement with the appropriate WHERE clauses at step 1516. This statement is then executed against the core database 144 at step 1520, using a COUNT statement to return a count of patients and not patient records. The response is returned to the Ember application 508 at step 1524 and the Ember application 508 updates the interface 1400 with the count of patients returned from the server at step 1528.

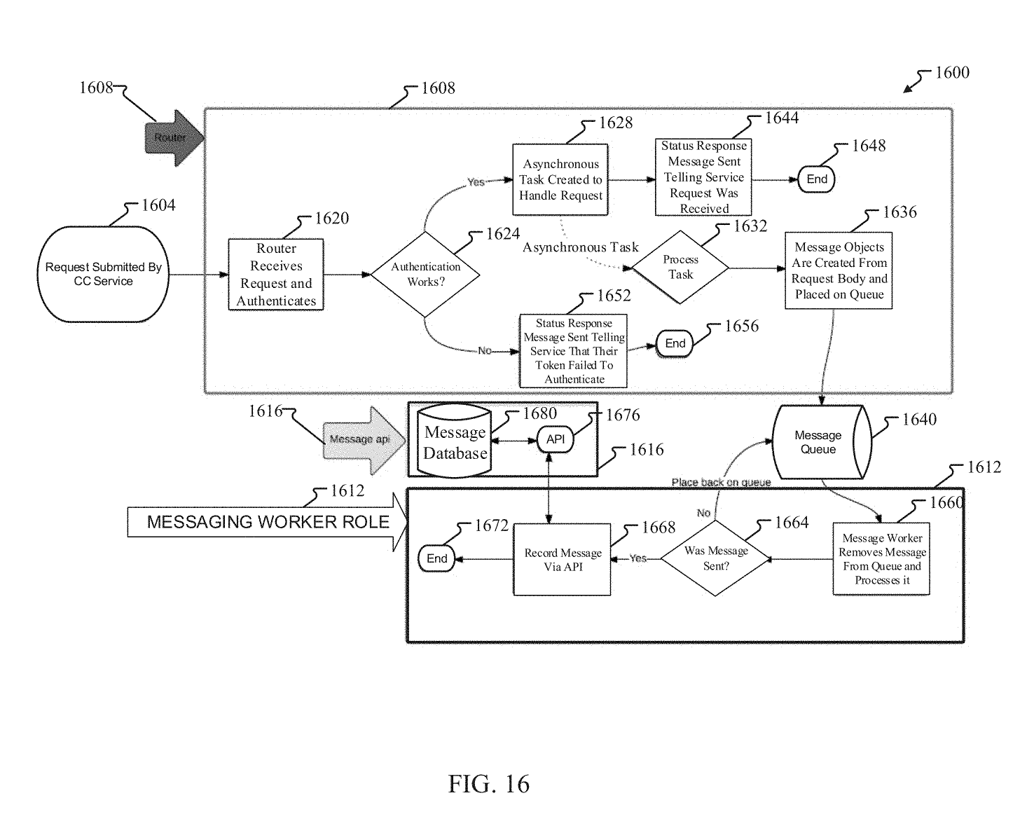

Referring now to FIG. 16, a method 1600 is discussed in accordance with embodiments of the present disclosure. Method 1600 generally illustrates how a message is submitted, processed, and eventually sent to a one or more patients. Method 1600 is in embodiments, performed by a device, such as the system 404. More specifically, one or more hardware and software components may be involved in performing method 1600. In one embodiment, the patient engagement and CRM platform API 104 may be involved in performing one or more of the steps of method 1600. The method 1600 may be executed as a set of computer-executable instructions executed by a computer system and encoded or stored on a computer-readable medium. Hereinafter, the method 1600 shall be explained with reference to systems, components, modules, software, etc. described with FIGS. 1-15.

Method 1600 may continuously flow in a loop, flow according to a timed event, or flow according to a change in an operating or status parameter. At step 1604, a message request may be submitted via the client engagement and CRM platform API 104 by one or more services 112 and/or one or more applications 108. For example, a FDW may click on button 1468 to send a group message to one or more recipients in a group. Accordingly, the request is sent to a router 1608, a messaging worker 1612, and eventually to the messaging API 1616. The messaging API 1616 may be the same as or similar to the messaging API 140. Alternatively, or in addition, the messaging API 161 may be a subset or a portion of the messaging API 140.

As depicted in FIG. 16, the router 1608 may receive the request and authenticate the message at step 1620. At step 1624, method 1600 proceeds to step 1628 if the authentication is successful. At step 1628, an asynchronous task is created to handle the messaging request. At step 1632 the message request is processed and message objects are created from the message body and placed on a messaging queue at step 1636. Simultaneously, or near simultaneously, a status response message may be sent to update the service request of the status and indicate that the request was received at which point step 1644 ends at step 1648. If the authentication is not successful at step 1620, method 1600 proceeds to step 1652 where a status response message is sent to the requesting service indicating that the token failed to authenticate and the method 1500 then ends at step 1656.

At step 1640, the message objects are received and put on a message queue. The messages in the message queue are then handled by one or more asynchronous workers 1612, such as a SideKiq asynchronous worker. That is, at step 1660, a message worker removes the message from the queue and processes the message to send the message. If the message was not successfully sent, the message may be returned to the message queue 1640. Otherwise, the method 1600 proceeds to step 1668 where the message may be recorded or logged via a messaging API 1616. At step 1672, the process with respect to the messaging worker role ends. At step 1676, the messaging API 1616 receives the message to be logged and places the message in the database 1680; the database 1680 may be the same as or a portion of the core database 144.

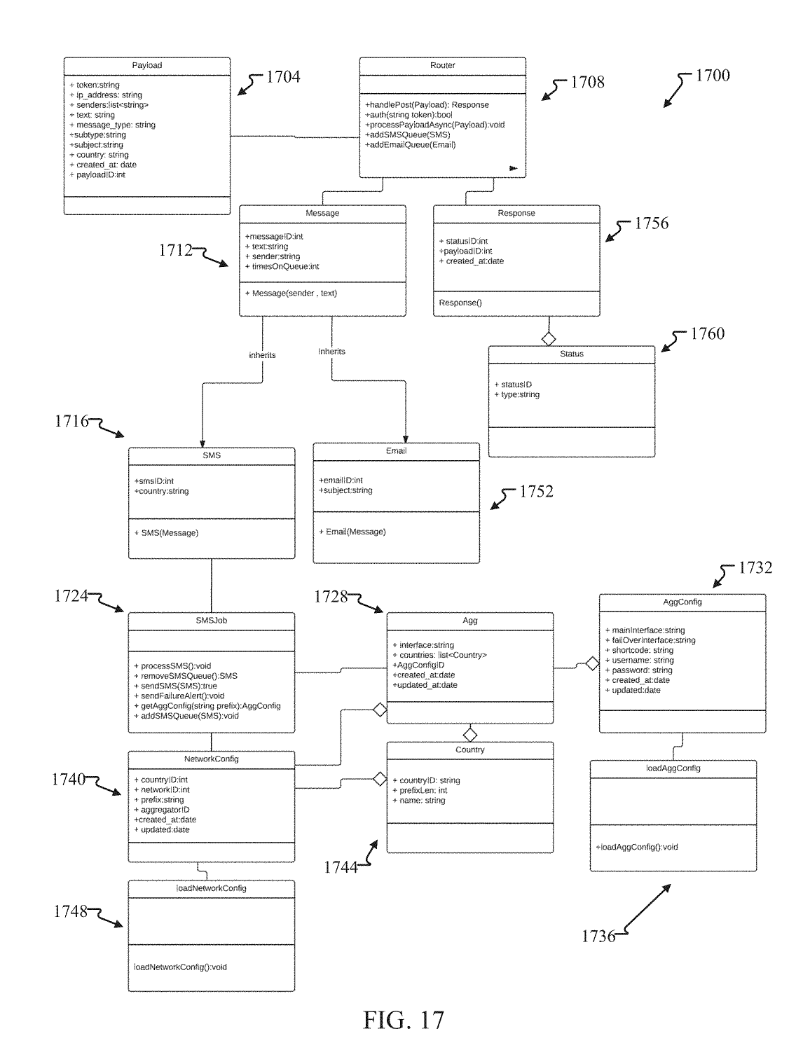

FIG. 17 depicts a UML diagram illustrating hierarchical inheritance of one or more classes utilized to send one or more messages as previously discussed. That is, a router 1708 may include a payload 1704, where the router is responsible for routing a message 172 and handling a response 1756. The message 1712 may inherit properties of SMS messages 1716 and email 1752, where the SMS 1716 may be associated with a job 1724 and aggregator 1728. The network configuration 1740 and request to load network configuration 1748 may be associated with both the aggregator 1728, where country information 1744 may impact the network configuration 1740. The aggregator 1728 may be associated with the aggregator configuration 1732 and request to load the configuration 1736.

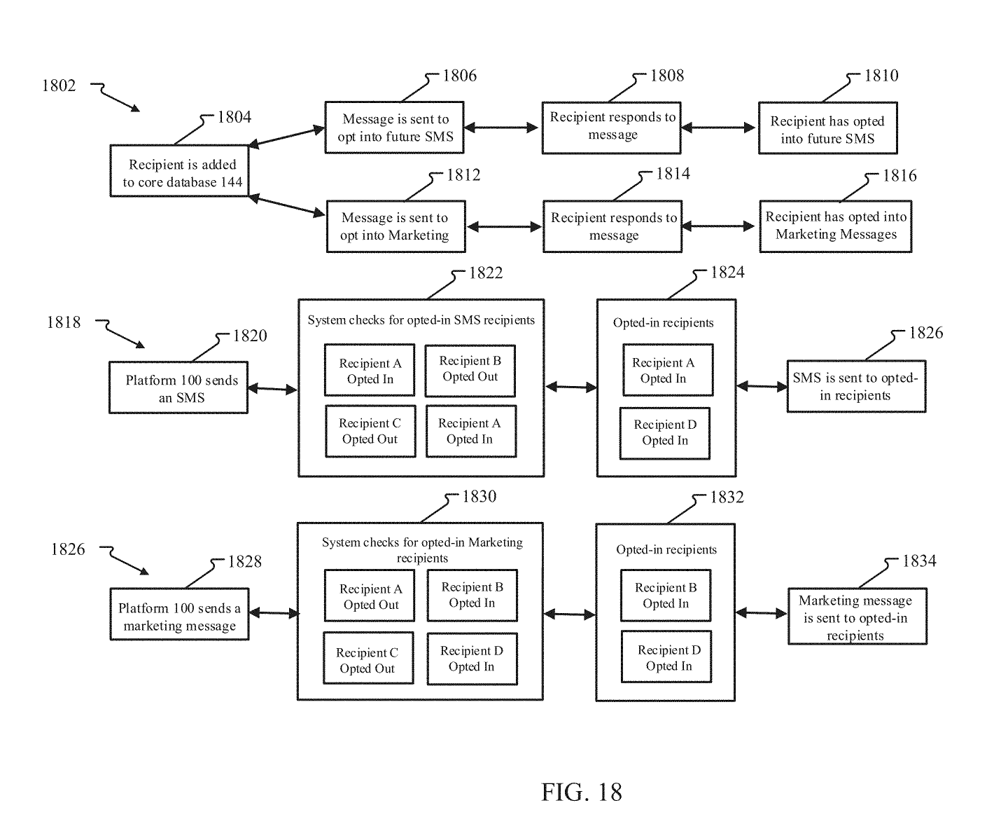

FIG. 18 depicts a process for opting into SMS and marketing communications utilized by the patient engagement and CRM platform 100. That is, details of an opt-in process 1802 that patients and any other users of the patient engagement and CRM platform 100 will undergo before communication with the patient and/or user is conducted. This diagram discusses SMS and marketing opt-in but also generically illustrates all opt-ins required by regulation. When a recipient is added to the patient engagement and CRM platform 100, the recipient is initially asked if they would like to opt into services provided by the patient engagement and CRM platform 100 including SMS and marketing services 1806/1812. Instructions on how to optin may be provided to the recipient in a paper or digital form. Telecommunication agencies, messaging platforms, and/or services may require the recipient to opt-in before any communication can be delivered to the recipient. Some Telecommunication agencies, messaging platforms, and/or services require a postscript message in the SMS to provide an option to opt-out. In whatever form is appropriate, the recipient opts into the communication at 1808/1814 and the opt-in preference is tracked in the core database 144 such that messages may be sent to the recipients in the future at 1810/1816. When sending a message to a group of recipients 1812/1826, the patient engagement and CRM platform 100 will first filter recipients based on their opt-in preferences at 1822/1830. The filtered recipients are selected for message delivery 1824/1832 and the message is sent to the recipient 1826/1834. Of course, in that FIG. 18 depicts a process for opting into SMS messaging, one skilled in the art will recognize that such a process is not limited to SMS, but may further include email, voice communication, and other forms of communication.

Any of the steps, functions, and operations discussed herein can be performed continuously and automatically.

The exemplary systems and methods of this disclosure have been described in relation to a the patient engagement and CRM platform 100. However, to avoid unnecessarily obscuring the present disclosure, the preceding description omits a number of known structures and devices. This omission is not to be construed as a limitation of the scope of the claimed disclosure. Specific details are set forth to provide an understanding of the present disclosure. It should however be appreciated that the present disclosure may be practiced in a variety of ways beyond the specific detail set forth herein.

Also, while the flowcharts have been discussed and illustrated in relation to a particular sequence of events, it should be appreciated that changes, additions, and omissions to this sequence can occur without materially affecting the operation of the disclosure.

A number of variations and modifications of the disclosure can be used. It would be possible to provide for some features of the disclosure without providing others.

In an embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the present disclosure includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the embodiments with reference to particular standards and protocols, the disclosure is not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease and\or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

While various embodiments of the present invention have been described in detail, it is apparent that modifications and alterations of those embodiments will occur to those skilled in the art. However, it is to be expressly understood that such modifications and alterations are within the scope and spirit of the present invention, as set forth in the following claims. Further, the invention(s) described herein is capable of other embodiments and of being practiced or of being carried out in various ways. In addition, it is to be understood that the phraseology and terminology used herein is for the purpose of description and should not be regarded as limiting.

What is claimed is:

1. A client engagement and customer relationship management platform comprising:
a core database including a plurality of patient records;
a client engagement and customer relationship management platform interface configured to provide access to the core database;
one or more interfaces configured to receive a selection of a filter and provide contact information for a group of patient records located at the core database and accessed via the client engagement and customer relationship management platform interface in accordance with the selected filter, wherein the group of patient records is defined by the selected filter; and
a messaging interface configured to receive the contact information for the group of patient records, receive a group message, and send the group message to one or more communication devices associated with each patient of the group of patients over a communication network utilizing the contact information.

2. The client engagement and customer relationship management platform of claim 1, further comprising:
an interface application configured to access the core database via the client engagement and customer relationship management platform interface, retrieve patient information from the core database, and store the retrieved patient information at a local storage device of which the interface application resides.

3. The client engagement and customer relationship management platform of claim 2, wherein the interface application is configured to determine the interface application is offline and store the retrieved patient information in an offline data store.

4. The client engagement and customer relationship management platform of claim 3, wherein the retrieved patient information is stored in an encrypted form on the local storage device of which the interface application resides.

5. The client engagement and customer relationship management platform of claim 4, wherein the interface application is configured to determine the interface application is online and store the retrieved patient information in an online data store.

6. The client engagement and customer relationship management platform of claim 3, wherein the interface application is configured to determine the interface application is online and move the retrieved patient information from the offline data store to an online data store.

7. The client engagement and customer relationship management platform of claim 6, wherein the interface application is determined to be online when the interface application can transfer a predetermined amount of information to the client engagement and customer relationship management platform within a predetermined amount of time.

8. The client engagement and customer relationship management platform of claim 2, wherein the client engagement and customer relationship management platform interface resides on a device separate from a device on which the interface application resides.

9. The client engagement and customer relationship management platform of claim 1, wherein the messaging interface is configured to receive a message from a communication device associated with a patient of the group of patients.

10. The client engagement and customer relationship management platform of claim 1, wherein the messaging interface is configured to communicate one or more of a Short Message Service (SMS) message, a real-time message, a message including audio content, or a message including video content.

11. A method, comprising:
receiving data at a server in a cloud network environment from various devices in a medical care facility providing health care services to at least one patient, wherein the data comprises medical data related to diagnosis and treatment of a health condition of the patient;
storing the data in a core database in the cloud;
extracting from the core database in the cloud, patient related information based on one or more criteria;
creating a group message to be sent to a plurality of patients in accordance with the one more criteria;
sending the group message to a first patient of the plurality of patients using a first communication medium; and
sending the group message to a second patient of the plurality of patients using a second communication medium, wherein the first communication medium is different from the second communication medium.

12. The method of claim 11, further comprising:
receiving an access request for patient information residing within the core database
retrieving the patient information from the core database; and
storing the retrieved patient information.

13. The method of claim 12, further comprising:
encrypting the retrieved patient information.

14. The method of claim 11, further comprising:
receiving a message from a communication device associated with a patient of the plurality of patients.

15. A computer-readable medium comprising one or more processor executable instructions, which when executed by a processor, perform the method of claim 11.

16. A method comprising:
accessing, by an interface application, a core database via a client engagement and customer relationship management platform;
retrieving patient information from the core database;
determining if the interface application has gone offline; and
storing the retrieved patient information in an offline data store located at a local storage area device of which the interface application resides.

17. The method of claim 16, further comprising;
determining that the interface application is online; and
moving the retrieved patient information from the offline data store to an online data store located at the local storage area device of which the interface application resides.

18. The method of claim 17, further comprising:
receiving a message at the interface application of the client engagement and customer relationship management platform; and
providing a message via the interface application to a messaging interface of the client engagement and customer relationship management platform.

19. The method of claim 16, further comprising:
providing a selection of a filter defining a group of patient records located at the core database;
receiving contact information for the group of patient records;
providing a group message; and
sending the group message to one or more communication devices associated with each patient of the group of patients over a communication network utilizing the contact information.

20. A computer-readable medium comprising one or more processor executable instructions, which when executed by a processor, perform the method of claim 16.
